(19) United States
(12) Patent Application Publication
(10) Pub. No.: US 2010/0021962 A1
(43) Pub. Date: Jan. 28, 2010

(54) Use of A Plastid-Lipid Associated Protein Promoter (PAP Promoter) For Heterologous Gene Expression

(75) Inventors: George Mather Sauer, Quedlinburg (DE); Ralf Flachmann, Quedlinburg (DE); Christel Renate Schopfer, Quedlinburg (DE)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 12/304,424

(22) PCT Filed: Jun. 12, 2007
(86) PCT No.: PCT/EP07/55756

§ 371 Date: Dec. 11, 2008

(30) Foreign Application Priority Data
Jun. 13, 2006 (EP) 06115339.1

(51) Int. Cl. A01H 5/00; C12N 15/82; C07H 21/04; C12P 23/00

(52) U.S. Cl. 435/67; 800/298; 800/278; 536/24.1

(57) ABSTRACT

Use of a plastid-lipid associated protein promoter (PAP promoter) for heterologous expression of genes in plants of the genus Tagetes.

The present invention relates to the use of a plastid-lipid associated protein promoter (PAP promoter) for heterologous gene expression, preferably for flower-specific expression of genes in plants of the genus Tagetes, to the genetically modified plants of the genus Tagetes, and to a process for producing biosynthetic products by cultivating the genetically modified plants,

### PRIOR ART

Various biosynthetic products such as, for example, fine chemicals, such as inter alia amino acids, vitamins, carotenoids, but also proteins, are produced by natural metabolic processes in cells and used in various branches of industry, including the human and animal food, cosmetics, feed, food and pharmaceutical industries.

These substances, which together are referred to as fine chemicals/proteins, comprise inter alia organic acids, both proteinogenic and non-proteinogenic amino acids, nucleotides and nucleosides, lipids and fatty acids, diols, carbohydrates, aromatic compounds, vitamins, carotenoids and cofactors, and proteins and enzymes. Production thereof on a large scale takes place in part by means of biotechnological processes using microorganisms which have been developed in order to produce and secrete large amounts of the particular desired substance.

Carotenoids are synthesized de novo in bacteria, algae, fungi and plants. In recent years there have been increasing attempts also to utilize plants as organisms for producing fine chemicals, especially for vitamins and carotenoids.

A natural mixture of the carotenoids lutein, zeaxanthin and violaxanthin is extracted for example from the flowers of marigold plants (Tagetes plants) as so-called oleoresin. This oleoresin is used both as constituent of dietary supplements and in the feed sector.

Lycopene from tomatoes is likewise used as dietary supplement, while phytoene is predominantly used in the cosmetics sector.

Ketocarotenoids, meaning carotenoids which comprise at least one keto group, such as, for example, astaxanthin, canthaxanthin, echinenone, 3-hydroxyechinenone, 3′-hydroxyechinenone, adonirubin and adonixanthin are natural antioxidants and pigments which are produced by some algae, plants and microorganisms as secondary metabolites.

Owing to their coloring properties, the ketocarotenoids and especially astaxanthin are used as pigmenting auxiliaries in livestock nutrition, especially in the rearing of trout, salmon and shrimps.

An economical biotechnological process for producing natural biosynthetic products and especially carotenoids is therefore of great importance.

WO 0032788 describes some carotenoid biosynthesis genes from plants of the genus Tagetes and discloses how genetically modified plants of the genus Tagetes could be produced in order to obtain different carotenoid profiles in the petals and thus to produce particular carotenoids specifically. For this it is necessary to overexpress some biosynthesis genes and to repress others.

For overexpression of the newly found carotenoid biosynthesis genes in plants of the genus Tagetes, WO 0032788 postulates the petal-specific promoter of the ketolase from Adonis vernalis.

WO 05019460 describes the use of promoters selected from EPSPS promoter, B-gene promoter, PDS promoter and CHRC promoter for expressing genes in Tagetes.

The promoters used to date cannot, however, satisfy all the requirements for high expression in Tagetes. There was consequently a need to provide promoters which better satisfy the requirements,

### DESCRIPTION OF THE INVENTION

The use of a plastid-lipid associated protein promoter (PAP promoter) for heterologous expression of genes in plants of the genus Tagetes has been found.

The use is particularly suitable for the flower-specific and particularly preferably for the petal-specific heterologous expression of genes in plants of the genus Tagetes. The BNPAPX promoter from Brassica is particularly suitable for accumulating novel ketocarotenoids, not previously present in Tagetes, in i) relatively high concentration and ii) preferably in the upper epidermis, covered with papillae, of the Tagetes petals. The promoter activity for the lower epidermis is, based on the accumulation of ketocarotenoids, to be categorized as weak.

A promoter means according to the invention a nucleic acid having expression activity, and thus means a nucleic acid which, functionally linked to a nucleic acid to be expressed, also referred to as gene hereinafter, regulates the expression, that is the transcription and the translation, of this nucleic acid or of this gene.

“Transcription” means according to the invention the process by which a complementary RNA molecule is produced starting from a DNA template. Proteins such-as RNA polymerase, so-called sigma factors and transcriptional regulator proteins are involved in this process. The synthesized RNA then serves as template in the translation process which then leads to the biosynthetically active protein.

A “functional linkage” means in this connection for example the sequential arrangement of one of the promoters of the invention and of a nucleic acid sequence to be expressed and, if appropriate, further regulatory elements such as, for example, a terminator in such a way that each of the regulatory elements is able to fulfil its function in the expression of the nucleic acid sequence. A direct linkage in the chemical sense is not absolutely necessary for this. Genetic control sequences such as, for example, enhancer sequences can also carry out their function on the target sequence from more remote positions or even from other DNA molecules. Arrangements in which the nucleic acid sequence to be expressed or the gene to be expressed is positioned behind (i.e. at the 3′ end) of the promoter sequence of the invention, so that the two sequences are covalently connected together, are preferred. It is preferred in this connection for the distance between the promoter sequence and the nucleic acid sequence to be expressed to be less than 200 base pairs, particularly preferably fewer than 100 base pairs, very particularly preferably fewer than 50 base pairs,

“Expression activity” means according to the invention the amount of protein formed by the promoter in a particular time, that is the expression rate.

“Specific expression activity” means according to the invention the amount of protein formed by the promoter in a particular time for each promoter.

In the case of a “caused expression activity” or “caused expression rate” in relation to a gene compared with the wild type, therefore, compared with the wild type the formation of a protein which was not present in this way in the wild type is caused.

In the case of an “increased expression activity” or “increased expression rate” in relation to a gene compared with the wild type, therefore, compared with the wild type the amount of the protein formed in a particular time is increased.

The formation rate at which a biosynthetically active protein is produced is a product of the rate of transcription and of translation. It is possible according to the invention to influence both rates, and thus to influence the rate of formation of products in a microorganism.

“Heterologous” gene expression means according to the invention that the promoter and the gene functionally linked thereto do not naturally occur in this arrangement in wild-type plants. Heterologous gene expression thus comprises the cases where the promoter or the gene to be expressed or both components do not occur naturally in the wild type of the corresponding plant, or else where both promoter and the gene to be expressed are naturally present in the wild-type plant but are on remote chromosomal positions, so that no functional linkage is present in the wild-type plant.

The term “wild type” or “wild-type plant” means according to the invention the corresponding initial plant of the genus Tagetes.

Depending on the context, the term “plant” may mean the initial plant (wild type) or a genetically modified plant of the invention of the genus Tagetes, or both.

“Wild type” preferably means for increasing or causing the expression activity or expression rate and for increasing the content of biosynthetic products the plant Tagetes erecta, especially the plant Tagetes erecta hybrid 50011 (WO 02012438), and the Tagetes erecta 13819 and the derivatives resulting therefrom by mutagenesis or cultivation, as reference organism.

A “PAP promoter” means promoters which naturally occur in plants such as cucumber, tomato, oilseed rape and others and which cause gene expression of plastid-associated proteins.

Preferred PAP promoters comprise
A1) the nucleic acid sequence SEQ. ID. NO. 9, 18 or 21 or
A2) a sequence derived from these sequences by substitution, insertion or deletion of nucleotides and having an identity of at least 60% at the nucleic acid level with the respective sequence SEQ. ID. NO. 9, 18 or 21, or
A3) a nucleic acid sequence which hybridizes with the nucleic acid sequence SEQ. ID. NO. 9, 18 or 21 under stringent conditions, or
A4) functionally equivalent fragments of the sequences under A1), A2) or A3)

The nucleic acid sequence SEQ. ID. NO, 9 represents a promoter sequence of the hypothetical plastid-lipid associated protein 2 from Lycopersicon esculentum.

The nucleic acid sequence SEQ. ID. NO. 18 represents a promoter sequence of a hypothetical plastid-lipid associated protein from Brassica napus.

The nucleic acid sequence SEQ. ID. NO. 21 represents a promoter sequence of a hypothetical plastid-lipid associated protein from Brassica napus.

The invention further relates to PAP promoters comprising a sequence derived from these sequences (SEQ. ID. NO. 9, 18 or 21) by substitution, insertion or deletion of nucleotides and having an identity of at least 60% at the nucleic acid level with the respective sequence SEQ. ID. NO. 9, 18 or 21.

Further natural examples of the invention of PAP promoters of the invention can be easily found for example from various organisms whose genomic sequence is known by comparisons of the identity of the nucleic acid sequences from databases with the sequences SEQ ID NO. 9, 18 or 21 described above.

Artificial PAP promoter sequences of the invention can easily be found starting from the sequences SEQ ID NO. 9, 18 or 21 by artificial variation and mutation, for example by substitution, insertion or deletion of nucleotides.

The following definition and conditions of the comparisons of identity and hybridization conditions apply to all nucleic acids, that is all promoters and genes of the description.

The term “substitution” means the exchange of one or more nucleotides for one or more nucleotides. “Deletion” is the replacement of a nucleotide by a direct linkage. Insertions are introductions of nucleotides into the nucleic acid sequence, where there is formal replacement of a direct linkage by one or more nucleotides.

Identity between two nucleic acids means the identity of the nucleotides over the whole length of the nucleic acid in each case, especially the identity calculated by comparison with the aid of the Vector NTI Suite 7.1 software from Informax (USA) using the Clustal method (Higgins D G, Sharp P M. Fast and sensitive multiple sequence alignments on a microcomputer. Comput Appl. Biosci. 1989 April;5(2):151-1) setting the following parameters:

Multiple Alignment Parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

Pairwise Alignment Parameter:

| FAST algorithm | on |
|---|---|
| K-tuple size | 1 |
| Gap penalty | 3 |
| Window size | 5 |
| Number of best diagonals | 5 |

A nucleic acid sequence having an identity of at least 60% with the sequence SEQ ID NO. 9 accordingly means a nucleic acid sequence which, on comparison of its sequence with the sequence SEQ ID NO. 9, in particular-in accordance with the above programming algorithm with the above set of parameters, shows an identity of at least 60%.

A nucleic acid sequence having an identity of at least 60% with the sequence SEQ ID NO. 18 accordingly means a nucleic acid sequence which, on comparison of its sequence with the sequence SEQ ID NO. 18 in particular in accordance with the above programming algorithm with the above set of parameters, shows an identity of at least 60%.

A nucleic acid sequence having an identity of at least 60% with the sequence SEQ ID NO. 21 accordingly means a nucleic acid sequence which, on comparison of its sequence with the sequence SEQ ID NO. 21, in particular in accordance with the above programming algorithm-with the above set of parameters, shows an identity of at least 60%.

Particularly preferred PAP promoters have an identity of at least 70%, preferably at least 80%, at least 90%, at least 92%, at least 95%, at least 96%, at least 97%, at least 98%, particularly preferably at least 99%, with the respective nucleic acid sequence SEQ. ID. NO. 9, 18 or 21.

Further natural examples of PAP promoters can further easily be found starting from the nucleic acid sequences described above, in particular starting from the sequences SEQ ID NO. 9, 18 or 21, from various organisms whose genomic sequence is unknown, by hybridization techniques in a manner known per se.

The invention therefore further relates to PAP promoters comprising a nucleic acid sequence which hybridizes with the nucleic acid sequence SEQ. ID. No. 9, 18 or 21 under stringent conditions. This nucleic acid sequence comprises at least 10, more preferably more than 12, 15, 30, 50 or particularly preferably more than 150 nucleotides.

“Hybridization” means the ability of a poly- or oligonucleotide to bind under stringent conditions to an almost complementary sequence, while nonspecific bindings between non-complementary partners do not occur under these conditions. For this, the sequences should preferably be 90-100% complementary. The property of complementary sequences being able to bind specifically to one another is made use of for example in the Northern or Southern blotting technique or in primer binding in PCR or RT-PCR.

The hybridization takes place according to the invention under stringent conditions. Such hybridization conditions are described for example in Sambrook, J., Fritsch, E. F., Maniatis, T., in: Molecular Cloning (A Laboratory Manual), 2^{nd} edition, Cold Spring Harbor Laboratory Press, 1989, pages 9.31-9.57 or in Current Protocols in Molecular Biology, John Wiley & Sons, N.Y. (1989), 6.3.1-6.3.6.

Stringent hybridization conditions mean in particular:

Overnight incubation at 42° C. in a solution consisting of 50% formamide, 5×SSC (750 mM NaCl, 75 mM trisodium citrate), 50 mM sodium phosphate (pH 7.6), 5× Denhardt's solution, 10% dextran sulfate and 20 g/ml denatured, sheared salmon sperm DNA, followed by washing the filters with 0.1×SSC at 65° C.

A “functionally equivalent fragment” means for promoters fragments which have essentially the same promoter activity as the initial sequence.

“Essentially identical” means a specific expression activity which displays at least 50%, preferably 60%, more preferably 70%, more preferably 80%, more preferably 90%, particularly preferably 95%, of the specific expression activity of the initial sequence.

“Fragments” mean partial sequences of the PAP promoters described by embodiment A1), A2) or A3). These fragments preferably have more than 10, but preferably more than 12, 15, 30, 50 or particularly preferably more than 150, connected nucleotides of the nucleic acid sequence SEQ. ID. NO. 1, 2 or 3.

It is particularly preferred to use the nucleic acid sequence SEQ. ID. NO. 9, 18 or 21 as PAP promoter, i.e. for expressing genes in plants of the genus Tagetes.

All the aforementioned PAP promoters can further be produced in a manner known per se by chemical synthesis from the nucleotide building blocks, such as, for example, by fragment condensation of individual overlapping, complementary nucleic acid building blocks of the double helix. The chemical synthesis of oligonucleotides can take place for example in a known manner by the phosphoamidite method (Voet, Voet, 2nd edition, Wiley Press New York, pp. 896-897). Addition of synthetic oligonucleotides and filling in of gaps using the Klenow fragment of DNA polymerase and ligation reactions, and general cloning methods are described in Sambrook et al. (1989), Molecular cloning: A laboratory manual, Cold Spring Harbor Laboratory Press.

It is possible with the promoters of the invention in principle for any gene to be expressed, in particular flower-specifically expressed, particularly preferably petal-specifically expressed, in plants of the genus Tagetes.

These genes to be expressed in plants of the genus Tagetes are also called “effect genes” hereinafter.

Preferred effect genes are for example genes from the biosynthetic pathway of odorous substances and flower colors whose expression or increased expression in plants of the genus Tagetes leads to an alteration of the odor and/or of the flower color of flowers of the plants of the genus Tagetes.

The biosynthesis of volatile odorous components, specifically in flowers, has been studied in recent years on various model organisms such as Clarkia breweri and Antirhinum majus L. Volatile odorous components are formed for example within the monoterpene and phenylpropane metabolism. Linalool is involved in the first case; methyleugenol, benzyl acetate, methyl benzoate and methyl salicylate are derived from the phenylpropanes.

Preferred genes for the biosynthesis of linalool, (iso)methyleugenol, benzyl acetate and methyl salicylate are selected from the group of nucleic acids encoding a linalool synthase (LIS), nucleic acids encoding an S-adenosyl-L-Met:(iso)-eugenol O-methyltransferase (IEMT), nucleic acids encoding an acetyl-CoA-benzyl alcohol acetyltransferase and nucleic acids encoding an S-adenosyl-L-Met:salicylic acid methyltransferase (SAMT). Nucleic acid sequences and protein sequences for the enzymatic activities mentioned are described in Dudareva et al. Plant Cell 8 (1996), 1137-1148; Wang et al. Plant Physiol. 114 (1997), 213-221 and Dudareva et al. Plant J. 14 (1998) 297-304).

Particularly preferred effect genes are genes from biosynthetic pathways of biosynthetic products which can naturally be produced in plants of the genus Tagetes, i.e. in the wild type or by genetic alteration of the wild type, can be produced in particular in flowers, can be produced particularly preferably in petals.

Preferred biosynthetic products are fine chemicals.

The term “fine chemical” is known in the art and includes compounds which are produced by an organism and are used in various branches of industry such as, for example but not restricted to, the pharmaceutical industry, the agriculture, cosmetics, food and feed industries. These compounds include organic acids such as, for example, tartaric acid, itaconic acid and diaminopimelic acid, and proteinogenic and non-proteinogenic amino acids, purine bases and pyrimidine bases, nucleosides and nucleotides (as described for example in Kuninaka, A. (1996) Nucleotides and related compounds, pp. 561-612, in Biotechnology vol. 6, Rehm et al., editors, VCH: Weinheim and the references present therein), lipids, saturated and unsaturated fatty acids (e.g. arachidonic acid), diols (e.g. propanediol and butanediol), carbohydrates (e.g. hyaluronic acid and trehalose), aromatic compounds (e.g. aromatic amines, vanillin and indigo), vitamins and cofactors (as described in Ullmann's Encyclopedia of Industrial Chemistry, volt A27, “Vitamins”, pp. 443-613 (1996) VCH: Weinheim and the references present therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held on Sep. 1-3, 1994 in Penang, Malaysia, AOCS Press (1995)), enzymes and all other chemicals described by Gutcho (1983) in Chemicals by Fermentation, Noyes Data Corporation, ISBN: 0818805086 and the references indicated therein. The metabolism and the uses of certain fine chemicals are explained further below.

I. Amino Acid Metabolism and Uses

The amino acids comprise the fundamental structural units of all proteins and are thus essential for normal cell functions. The term “amino acid” is known in the art. The proteinogenic amino acids, of which there are 20 types, serve as structural units for proteins, in which they are linked together by peptide bonds, whereas the non-proteinogenic amino acids (of which hundreds are known) usually do not occur in proteins (see Ullmann's Encyclopedia of Industrial Chemistry, vol. A2, pp. 57-97 VCH: Weinheim (1985)). The amino acids may be in the D or L configuration, although L-amino acids are usually the only type found in naturally occurring proteins. Biosynthetic and degradation pathways of each of the 20 proteinogenic amino acids are well characterized both in prokaryotic and in eukaryotic cells (see, for example, Stryer, L. Biochemistry, 3rd edition, pp. 578-590 (1988)). The “essential” amino acids (histidine, isoleucine, leucine, lysine, methionine, phenylalanine, threonine, tryptophan and valine), so-called because they must, owing to the complexity of their biosynthesis, be taken in with the diet, are converted by simple biosynthetic pathways into the other 11 “nonessential” amino acids (alanine, arginine, asparagine, aspartate, cysteine, glutamate, glutamine, glycine, proline, serine and tyrosine). Higher animals have the ability to synthesize some of these amino acids, but the essential amino acids must be taken in with the food in order for normal protein synthesis to take place.

Apart from their function in protein biosynthesis, these amino acids are chemicals of interest per se, and it has been found that many have uses in various applications in the food, feed, chemicals, cosmetics, agriculture and pharmaceutical industries. Lysine is an important amino acid not only for human nutrition but also for monogastric species such as poultry and pigs. Glutamate is used most frequently as flavor additive (monosodium glutamate, MSG) and widely in the food industry, as well as aspartate, phenylalanine, glycine and cysteine. Glycine, L-methionine and tryptophan are all used in the pharmaceutical industry. Glutamine, valine, leucine, isoleucine, histidine, arginine, proline, serine and alanine are used in the pharmaceutical industry and the cosmetics industry. Threonine, tryptophan and D-L-methionine are widely used feed additives (Leuchtenberger, W. (1996) Amino acids—technical production and use, pp. 466-502in Rehm et al., (editors) Biotechnology vol. 6, chapter 14a, VCH: Weinheim). It has been found that these amino acids are additionally suitable as precursors for synthesizing synthetic amino acids and proteins such as N-aaetylcysteine, S-carboxymethyl-L-cysteine, (S)-5-hydroxytryptophan and other substances described in Ullmann's Encyclopedia of Industrial Chemistry, vol. A2, pp. 57-97, VCH, Weinheim, 1985.

The biosynthesis of these natural amino acids in organisms able to produce them, for example bacteria, has been well characterized (for a review of bacterial amino acid biosynthesis and its regulation, see Umbarger, H. E. (1978) Ann. Rev. Biochem. 47; 533-606). Glutamate is synthesized by reductive amination of α-ketoglutarate, an intermediate in the citric acid cycle. Glutamine, proline and arginine are each generated successively from glutamate. Biosynthesis of serine takes place in a three-step method and starts with 3-phosphoglycerate (an intermediate of glycolysis) and yields this amino acid after oxidation, transamination and hydrolysis steps. Cysteine and glycine are each produced from serine, the former by condensation of homocysteine with serine, and the latter by transfer of the side-chain β-carbon atom to tetrahydrofolate in a reaction catalyzed by serine transhydroxymethylase. Phenylalanine and tyrosine are synthesized from the precursors of the glycolysis and pentose phosphate pathways, erythrose 4-phosphate and phosphenolpyruvate in a 9-step biosynthetic pathway which differs only in the last two steps after the synthesis of prephenate. Tryptophan is likewise produced from these two starting molecules, but its synthesis takes place in an 11-step pathway. Tyrosine can also be produced from phenylalanine in a reaction catalyzed by phenylalanine hydroxylase. Alanine, valine and leucine are each biosynthetic products of pyruvate, the final product of glycolysis. Aspartate is formed from oxalapetate, an intermediate of the citrate cycle. Asparagine, methionine, threonine and lysine are each produced by conversion of aspartate. Isoleucine is formed from threonine. Histidine is formed in a complex 9-step pathway from 5-phosphoribosyl 1-pyrophosphate, an activated sugar.

Amino acids whose quantity exceeds the protein biosynthesis requirement of the cell cannot be stored and are instead degraded, so that intermediates are provided for the main metabolic pathways of the cell (for a review, see Stryer, L., Biochemistry, 3rd edition, chapter 21 “Amino Acid Degradation and the Urea Cycle”; pp. 495-516 (1988)). Although the cell is able to convert unwanted amino acids into useful metabolic intermediates, amino acid production is costly in terms of the energy, the precursor molecules and the enzymes required for their synthesis. It is therefore not surprising that amino acid biosynthesis is regulated by feedback inhibition, where the presence of a particular amino acid slows down or entirely terminates its own production (for a review of the feedback mechanism in amino acid biosynthetic pathways, see Stryer, L., Biochemistry, 3rd edition, chapter 24; “Biosynthesis of Amino Acids and Heme”, pp. 575-600 (1988)). The output of a particular amino acid is therefore restricted by the quantity of this amino acid in the cell.

II. Vitamins, Carotenoids, Cofactors and Nutraceutical Metabolism, and Uses

Vitamins, carotenoids, cofactors and nutraceuticals comprise a further group of molecules. Higher animals have lost the ability to synthesize these and therefore need to take them in, although they are easily synthesized by other organisms such as bacteria. These molecules are either biologically active molecules per se or precursors of biologically active substances which serve as electron carriers or intermediates in a number of metabolic pathways. These compounds have, besides their nutritional value, also a significant industrial value as coloring agents, antioxidants and catalysts or other processing aids. (For a review of the structure, activity and industrial applications of these compounds, see, for example, Ullmann's Encyclopedia of Industrial Chemistry, “Vitamins”, vol. A27, pp. 443-613, VCH: Weinheim, 1996). The term “vitamin” is known in the art and includes nutrients which are required by an organism for normal functioning, but cannot be synthesized by this organism itself. The group of vitamins may include cofactors and nutraceutical compounds. The term “cofactor” includes non-protein compounds which are necessary for the occurrence of normal enzymic activity. These compounds may be organic or inorganic; the cofactor molecules of the invention are preferably organic. The term “nutraceutical” includes food additives which promote health in plants and animals, especially in humans. Examples of such molecules are vitamins, antioxidants and likewise certain lipids (e.g. polyunsaturated fatty acids).

Preferred fine chemicals or biosynthetic products which can be produced in plants of the genus Tagetes, especially in petals of the flowers of the plants of the genus Tagetes, are carotenoids such as, for example, phytoene, lycopene, beta-carotene, lutein, zeaxanthin, astaxanthin, canthaxanthin, echinenone, 3-hydroxyechinenone, 3′-hydroxyechinenone, adonirubin, violaxanthin and adonixanthin.

Particularly preferred carotenoids are ketocarotenoids such as, for example, astaxanthin, canthaxanthin, echinenone, 3-hydroxyechinenone, 3′-hydroxyechinenone, adonirubin and adonixanthin.

Biosynthesis of these molecules in organisms able to produce them, such as bacteria, has been characterized in detail (Ullmann's Encyclopedia of Industrial Chemistry, “Vitamins”, vol. A27, pp. 443-613, VCH: Weinheim, 1996, Michal, G. (1999) Biochemical Pathways. An Atlas of Biochemistry and Molecular Biology, John Wiley & Sons; Ong, A. S., Niki, E. and Packer, L. (1995) “Nutrition, Lipids, Health and Disease” Proceedings of the UNESCO/Confederation of Scientific and Technological Associations in Malaysia and the Society for free Radical Research—Asia, held on Sep. 1-3, 1994,in Penang, Malaysia, AOCS Press, Champaign, IL X, 374 S).

Thiamine (vitamin B₁) is formed by chemical coupling of pyrimidine and thiazole units. Riboflavin (vitamin B₂) is synthesized from guanosine 5′-triphosphate (GTP) and ribose 5′-phosphate. Riboflavin in turn is employed for the synthesis of flavin mononucleotide (FMN) and flavin-adenine dinucleotide (FAD). The family of compounds referred to jointly as “vitamin B6” (e.g. pyridoxine, pyridoxamine, pyridoxal 5′-phosphate and the commercially used pyridoxine hydrochloride) are all derivatives of the common structural unit 5-hydroxy-6-methylpyridine. Pantothenate (pantothenic acid, R-(+)-N-(2,4-dihydroxy-3,3-dimethyl-1-oxobutyl)-β-alanine) can be produced either by chemical synthesis or by fermentation. The last steps in pantothenate biosynthesis consist of ATP-driven condensation of β-alanine and pantoic acid. The enzymes responsible for the biosynthetic steps for conversion into pantoic acid, into β-alanine and for condensation to pantothenic acid are known. The metabolically active form of pantothenate is coenzyme A, whose biosynthesis proceeds through 5 enzymatic steps. Pantothenate, pyridoxal 5-phosphate, cysteine and ATP are the precursors of coenzyme A. These enzymes catalyze not only the formation of pantothenate but also the production of (R)-pantoic acid, (R)-pantolactone, (R)-panthenol (provitamin B5), pantethein (and its derivatives) and coenzyme A.

The biosynthesis of biotin from the precursor molecule pimeloyl-CoA in microorganisms has been investigated in detail, and several of the genes involved have been identified. It has emerged that many of the corresponding proteins are involved in Fe cluster synthesis and belong to the class of nifS proteins. Lipoic acid is derived from octanonic acid and serves as coenzyme in energy metabolism, where it becomes a constituent of the pyruvate dehydrogenase complex and of the α-ketoglutarate dehydrogenase complex. The folates are a group of substances which are all derived from folic acid, which in turn is derived from L-glutamic acid, p-aminobenzoic acid and 6-methylpterin. The biosynthesis of folic acid and its derivatives starting from the metabolic intermediates guanosine 5′-triphosphate (GTP), L-glutamic acid and p-aminobenzoic acid has been investigated in detail in certain microorganisms.

Corrinoids (such as the cobalamins and in particular vitamin B₁₂) and the porphyrins belong to a group of chemicals which are distinguished by a tetrapyrrole ring system. The biosynthesis of vitamin B₁₂ is so complex that it has not yet been completely characterized, but most of the enzymes and substrates involved are now known. Nicotinic acid (nicotinate) and nicotinamide are pyridine derivatives, which are also referred to as “niacin”. Niacin is the precursor of the important coenzymes NAD (nicotinamide-adenine dinucleotide) and NADP (nicotinamide-adenine dinucleotide phosphate) and their reduced forms.

The production of these compounds on the industrial'scale is based for the most part on cell-free chemical syntheses, although some of these chemicals have likewise been produced by large-scale culturing of microorganisms, such as riboflavin, vitamin B₆, pantothenate and biotin. Only vitamin B₁₂ is produced solely by fermentation, because of the complexity of its synthesis. In vitro methods require a considerable expenditure of materials and time and frequently of high costs.

III. Purine, Pyrimidine, Nucleoside and Nucleotide Metabolism and, Uses

Genes for purine and pyrimidine metabolism and their corresponding proteins are important targets for the therapy of neoplastic diseases and viral infections. The term “purine” or “pyrimidine” comprises nitrogenous bases which are a constituent of nucleic acids, coenzymes and nucleotides. The term “nucleotide” comprises the fundamental structural units of nucleic acid molecules, which include a nitrogenous base, a pentose sugar (the sugar in RNA is ribose, and the sugar in DNA is D-deoxyribose) and phosphoric acid. The term “nucleoside” comprises molecules which serve as precursors of nucleotides but which, in contrast to nucleotides, have no phosphoric acid unit. It is possible by inhibiting the biosynthesis of these molecules or their mobilization for formation of nucleic acid molecules to inhibit RNA and DNA synthesis; targeted inhibition of this activity in carcinogenic cells allows the ability of tumor cells to divide and replicate to be inhibited.

There are also nucleotides which do not form nucleic acid molecules but serve as energy stores (i.e. AMP) or as coenzymes (i.e. FAD and NAD).

Several publications have described the use of these chemicals for these medical indications, where purine and/or pyrimidine metabolism is influenced (e.g. Christopherson, R. I. and Lyons, S. D. (1990) “Potent inhibitors of de novo pyrimidine and purine biosynthesis as chemotherapeutic agents”, Med. Res. Reviews 10: 505-548). Investigations on enzymes involved in purine and pyrimidine metabolism have concentrated on the development of novel medicaments which can be used for example as immunosuppressants or antiproliferatives (Smith, J. L. “Enzymes in Nucleotide Synthesis” Curr. Opin. Struct. Biol. 5 (1995) 752-757; Biochem. Soc. Transact. 23 (1995) 877-902). Purine and pyrimidine bases, nucleosides and nucleotides have, however, also other possible uses: as intermediates in the biosynthesis of various fine chemicals (e.g. thiamine, S-adenosylmethionine, folates or riboflavin), as energy carriers for the cell (e.g. ATP or GTP) and for chemicals themselves, are commonly used as flavor enhancers (e.g. IMP or GMP) or for many medical applications (see, for example, Kuninaka, A., (1996) “Nucleotides and Related Compounds” in Biotechnology, vol. 6, Rehm et al., editors VCH: Weinheim, pp. 561-612). Enzymes involved in purine, pyridine, nucleoside or nucleotide metabolism are also increasingly serving as targets for the development of chemicals for crop protection, including fungicides, herbicides and insecticides.

The metabolism of these compounds in bacteria has been characterized (for reviews, see, for example, Zalkin, H. and Dixon, J. E. (1992) “De novo purin nucleotide biosynthesis” in Progress in Nucleic Acids Research and Molecular biology, vol. 42, Academic Press,. pp. 259-287; and Michal, G. (1999) “Nucleotides and Nucleosides”; chapter 8 in: Biochemical Pathways: An Atlas of Biochemistry and Molecular Biology, Wiley, New York). Purine metabolism, which is the subject of intensive research, is essential for normal functioning of the cell. Impaired purine metabolism in higher animals may cause severe disorders, e.g. gout. The purine nucleotides are synthesized over a number of steps via the intermediate compound inosine 5′-phosphate (IMP) from ribose 5-phosphate, leading to production of guanosine 5′-monophosphate (GMP) or adenosine 5′-monophosphate (AMP), from which the triphosphate forms, which are used as nucleotides, can easily be prepared. These compounds are also used as energy stores, so that their degradation provides energy for many different biochemical processes in the cell. Pyrimidine biosynthesis takes place via the formation of uridine. 5′-monophosphate (UMP) from ribose 5-phosphate. UMP in turn is converted into cytidine 5′-triphosphate (CTP). The deoxy forms of all nucleotides are prepared in a one-step reduction reaction from the diphosphate ribose form of the nucleotide to give the diphosphate deoxyribose form of the nucleotide. After phosphorylation, these molecules are able to take part in DNA synthesis.

IV. Trehalose Metabolism and Uses

Trehalose consists of two glucose molecules which are linked together via an α,α-1,1 linkage. It is commonly used in the food industry as sweetener, as additive to dried or 25 frozen foods and in beverages. However, it is also used in the pharmaceutical industry, the cosmetics and biotechnology industry (see, for example, Nishimoto et al., (1998) U.S. Pat. No. 5,759,610; Singer, M. A. and Lindquist, S. Trends Biotech. 16 (1998) 460-467; Paiva, C. L. A. and Panek, A. D. Biotech Ann. Rev. 2 (1996) 293-314; and Shiosaka, M. J. Japan 172 (1997) 97-102). Trehalose is produced by enzymes of many microorganisms and is released in a natural way into the surrounding medium, from which it can be isolated by methods known in the art.

Particularly preferred biosynthetic products are selected from the group of organic acids, proteins, nucleotides and nucleosides, both proteinogenic and non-proteinogenic amino acids, lipids and fatty acids, diols, carbohydrates, aromatic compounds, vitamins and cofactors, enzymes and proteins.

Preferred organic acids are tartaric acid, itaconic acid and diaminopimelic acid.

Preferred nucleosides and nucleotides are described for example in Kuninaka, A. (1996) Nucleotides and related compounds, pp. 561-612, in Biotechnology, vol. 6, Rehm et al., editors VCH: Weinheim and references present therein.

Preferred biosynthetic products are additionally lipids, saturated and unsaturated fatty acids such as, for example, arachidonic acid, diols such as, for example, propanediol and butanediol, carbohydrates such as, for example, hyaluronic acid and trehalose, aromatic compounds such as, for example, aromatic amines, vanillin and indigo, vitamins and cofactors as described for example in Ullmann's Encyclopedia of Industrial Chemistry, vol. A27, “Vitamins”, pp. 443-613 (1996) VCH: Weinheim and the references present therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held on Sep. 1-3, 1994 in Penang, Malaysia, AOCS Press (1995)), enzymes, polyketides (Cane et al. (1998) Science 282: 63-68) and all other chemicals described by Gutcho (1983) in Chemicals by Fermentation, Noyes Data Corporation, ISBN: 0818805086 and the references indicated therein.

Particularly preferred genes expressed with the promoters of the invention in plants of the genus Tagetes are accordingly genes selected from the group of nucleic acids encoding a protein from the biosynthetic pathway of proteinogenic and non-proteinogenic amino acids, nucleic acids encoding a protein from the biosynthetic pathway of nucleotides and nucleosides, nucleic acids encoding a protein from the biosynthetic pathway of organic acids, nucleic acids encoding a protein from the biosynthetic pathway of lipids and fatty acids, nucleic acids encoding a protein from the biosynthetic pathway of diols, nucleic acids encoding a protein from the biosynthetic pathway of carbohydrates, nucleic acids encoding a protein from the biosynthetic pathway of aromatic compounds, nucleic acids encoding a protein from the biosynthetic pathway of vitamins, nucleic acids encoding a protein from the biosynthetic pathway of carotenoids, especially ketocarotenoids, nucleic acids encoding a protein from the biosynthetic pathway of cofactors and nucleic acids encoding a protein from the biosynthetic pathway of enzymes.

Preferred fine chemicals or biosynthetic products which can be produced in plants of the genus Tagetes, especially in petals of the flowers of the plants of the genus Tagetes, are carotenoids such as, for example, phytoene, lycopene, beta-carotene, lutein, zeaxanthin, astaxanthin, canthaxanthin, echinenone, 3-hydroxyechinenone, 3′-hydroxyechinenone, adonirubin, violaxanthin and adonixanthin.

Particularly preferred carotenoids are ketocarotenoids such as, for example, astaxanthin, canthaxanthin, echinenone, 3-hydroxyechinenone, 3′-hydroxyechinenone, adonirubin and adonixanthin.

Very particularly preferred genes expressed with the promoters of the invention in plants of the genus Tagetes are accordingly genes which encode proteins from the biosynthetic pathway of carotenoids.

Particularly preferred genes are selected from the group of nucleic acids encoding a ketolase, nucleic acids encoding a β-hydroxylase, nucleic acids encoding a β-cyclase, nucleic acids encoding an ε-cyclase, nucleic acids encoding a zeaxanthin epoxidase, nucleic acids encoding an antheraxanthin epoxidase, nucleic acids encoding a neoxanthin synthase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl-diphosphate Δ-isomerase, nucleic acids encoding a geranyl-diphosphate synthase, nucleic acids encoding a farnesyl-diphosphate synthase, nucleic acids encoding a geranyl-geranyl-diphosphate synthase, nucleic acids encoding a phytoene synthase, nucleic acids encoding a phytoene desaturase (phytoene dehydrogenase), nucleic acids encoding a prephytoene synthase, nucleic acids encoding a zeta-carotene desaturase, nucleic acids encoding a crtISO protein, nucleic acids encoding a 4-diphosphocytidyl-2-C-methyl-D-erythritol synthase, nucleic acids encoding a 4-diphosphocytidyl-2-C-methyl-D-erythritol kinase, nucleic acids encoding a 2-methyl-D-erythritol-2,4-cyclodiphosphate synthase, nucleic acids encoding a hydroxymethylbutenyl-diphosphate synthase, nucleic acids encoding an FtsZ protein and nucleic acids encoding an MinD protein.

A ketolase means a protein which has the enzymatic activity of introducing a keto group on the optionally substituted β-ionone ring of carotenoids.

Ketolase means in particular a protein which has the enzymatic activity of converting β-carotene into canthaxanthin.

Examples of nucleic acids encoding a ketolase, and the corresponding ketolases, are for example sequences from

Haematoccus pluvialis, especially from Haematoccus pluvialis Flotow em. Wille (Accession NO: X86782, SEQ ID No. 24).

Haematoccus pluvialis, NIES-144 (Accession NO: D45881)

Agrobacterium aurantiacum (Accession NO: D584209)

Alicaligenes spec. (Accession NO: D58422)

Paracoccus marcusii (Accession NO: Y15112)

Synechocystis sp. Strain PC6803 (Accession NO: NP442491)

Bradyrhizobium sp. (Accession NO: AF218415)

Nostoc sp, Strain PCC7120 (Accession NO: AP003592, BAB74888)

Haematococcus pluvialis (Accession NO: AF534876, AAN03484)

Paracoccus sp. MBIC1143 (Accession NO: 058420, P54972)

Brevundimonas aurantiaca (Accession NO: AY166610, AAN86030)

Nodularia spumigena NSOR10 (Accession NO: AY210783, AA064399)

Nostoc punctiforme ATCC 29133 (Accession NO: NZ_AABC01000195, ZP_{—}00111258);

Nostoc punctiforme ATCC 29133

Deinococcus radiodurans R1 (Accession NO: E75561, AE001872)

Synechococcus sp. WH 8102, nucleic acid: Acc. No. NZ_AABD01000001, base pair 1,354,725-1,355,528

A β-cyclase means a protein which has the enzymatic activity of converting a terminal linear lycopene residue into a β-ionone ring.

A β-cyclase means in particular a protein which has the enzymatic activity of converting γ-carotene into β-carotene.

Examples of β-cyclase genes are nucleic acids encoding a β-cyclase from tomato (Accession X86452), and β-cyclases of the following Accession Numbers:

| | |
|---|---|
| S66350 | lycopene beta-cyclase (EC 5.5.1.-) - tomato |
| CAA60119 | lycopene synthase [Capsicum annuum] |
| S66349 | lycopene beta-cyclase (EC 5.5.1.-) - common tobacco |
| CAA57386 | lycopene cyclase [Nicotiana tabacum] |
| AAM21152 | lycopene beta-cyclase [Citrus sinensis] |
| AAD38049 | lycopene cyclase [Citrus × paradisi] |
| AAN86060 | lycopene cyclase [Citrus unshiu] |
| AAF44700 | lycopene beta-cyclase [Citrus sinensis] |
| AAK07430 | lycopene beta-cyclase [Adonis palaestina] |
| AAG10429 | beta cyclase [Tagetes erecta] |
| AAA81880 | lycopene cyclase |
| AAB53337 | Lycopene beta cyclase |
| AAL92175 | beta-lycopene cyclase [Sandersonia aurantiaca] |
| CAA67331 | lycopene cyclase [Narcissus pseudonarcissus] |
| AAM45381 | beta cyclase [Tagetes erecta] |
| AAO18661 | lycopene beta-cyclase [Zea mays] |
| AAG21133 | chromoplast-specific lycopene beta-cyclase [Lycopersicon |
| | esculentum] |
| AAF18989 | lycopene beta-cyclase [Daucus carota] |
| ZP_001140 | hypothetical protein [Prochlorococcus marinus str. |
| | MIT9313] |
| ZP_001050 | hypothetical protein [Prochlorococcus marinus subsp. |
| | pastoris str. CCMP1378] |
| ZP_001046 | hypothetical protein [Prochlorococcus marinus subsp. |
| | pastoris str. CCMP1378] |
| ZP_001134 | hypothetical protein [Prochlorococcus marinus str. |
| | MIT9313] |
| ZP_001150 | hypothetical protein [Synechococcus sp. WH 8102] |
| AAF10377 | lycopene cyclase [Deinococcus radiodurans] |
| BAA29250 | 393aa long hypothetical protein [Pyrococcus horikoshii] |
| BAC77673 | lycopene beta-monocyclase [marine bacterium P99-3] |
| AAL01999 | lycopene cyclase [Xanthobacter sp. Py2] |
| ZP_000190 | hypothetical protein [Chloroflexus aurantiacus] |
| ZP_000941 | hypothetical protein [Novosphingobium aromaticivorans] |
| AAF78200 | lycopene cyclase [Bradyrhizobium sp. ORS278] |
| BAB79602 | crtY [Pantoea agglomerans pv. milletiae] |
| CAA64855 | lycopene cyclase [Streptomyces griseus] |
| AAA21262 | dycopene cyclase [Pantoea agglomerans] |
| C37802 | crtY protein - Erwinia uredovora |
| BAB79602 | crtY [Pantoea agglomerans pv. milletiae] |
| AAA64980 | lycopene cyclase [Pantoea agglomerans] |
| AAC44851 | lycopene cyclase |
| BAA09593 | Lycopene cyclase [Paracoccus sp. MBIC1143] |
| ZP_000941 | hypothetical protein [Novosphingobium aromaticivorans] |
| CAB56061 | lycopene beta-cyclase [Paracoccus marcusii] |
| BAA20275 | lycopene cyclase [Erythrobacter longus] |
| ZP_000570 | hypothetical protein [Thermobifida fusca] |
| ZP_000190 | hypothetical protein [Chloroflexus aurantiacus] |
| AAK07430 | lycopene beta-cyclase [Adonis palaestina] |
| CAA67331 | lycopene cyclase [Narcissus pseudonarcissus] |
| AAB53337 | Lycopene beta cyclase |
| BAC77673 | lycopene beta-monocyclase [marine bacterium P99-3] |

A particularly preferred β-cyclase is moreover the chromoplast-specific β-cyclase from tomato (AAG21133).

A hydroxylase means a protein which has the enzymatic activity of introducing a hydroxy group on the optionally substituted β-ionone ring of carotenoids.

A hydroxylase means in particular a protein which has the enzymatic activity of converting β-carotene into zeaxanthin or canthaxanthin into astaxanthin.

Examples of a hydroxylase gene are:

a nucleic acid encoding a hydroxylase from Haematococcus pluvialis, Accession AX038729, WO 0061764); and hydroxylases of the following Accession Numbers.

|emb|CAB55626.1, CAA70427.1, CAA70888.1, CAB55625.1, AF499108_{—}1, AF315289_{—}1, AF296158_{—}1, AAC49443.1, NP_{—}194300.1, NP_{—}200070.1, AAG10430.1, CAC06712.1, AAM88619.1, CAC95130.11 AAL80006.1, AF162276_{—}1, AA053295.1, MN85601.1, CRTZ_ERWHE, CRTZ_PANAN, BAB79605.1, CRTZ_ALCSP, CRTZ_AGRAU, CAB56060.1, ZP_{—}00094836.1, AAC44852.1, BAC77670.1, NP_{—}745389.1, NP_{—}344225.1, NP_{—}849490.1 ZP_{—}00087019.1, NP_{—}503072.1, NP_{—}852012.1, NP_{—}115929.1, ZP_{—}00013255.1

A particularly preferred hydroxylase is moreover the hydroxylase from tomato (Accession Y14810₇ CrtR-b2).

An HMG-CoA reductase means a protein which has the enzymatic activity of converting 3-hydroxy-3-methylglutaryl-coenzyme A into mevalonate.

An (E)-4-hydroxy-3-methylbut-2-enyl-diphosphate reductase means a protein which has the enzymatic activity of converting (E)4-hydroxy-3-methylbut-2-enyl diphosphate into isopentenyl diphosphate and dimethylallyl diphosphates.

A 1-deoxy-D-xylose-5-phosphate synthase means a protein which has the enzymatic activity of converting hydroxyethyl-ThPP and glyceraldehyde 3-phosphate into 1-deoxy-D-xylose 5-phosphate:

A 1-deoxy-D-xylose-5-phosphate reductoisomerase means a protein which has the enzymatic activity of converting 1-deoxy-D-xylose 5-phosphate into 2-C-methyl-D-erythritol 4-phosphate.

An isopentenyl-diphosphate Δ-isomerase means a protein which has the enzymatic activity of converting isopentenyl diphosphate into dimethylallyl phosphate.

A geranyl-diphosphate synthase means a protein which has the enzymatic activity of converting isopentenyl diphosphate and dimethylallyl phosphate into geranyl diphosphate.

A farnesyl-diphosphate synthase means a protein which has the enzymatic activity of sequentially converting 2 molecules of isopentenyl diphosphate with dimethylallyl diphosphate and the resulting geranyl diphosphate into farnesyl diphosphate.

A geranyl-geranyl-diphosphate synthase means a protein which has the enzymatic activity of converting farnesyl diphosphate and isopentenyl diphosphate into geranyl-geranyl diphosphate.

A phytoene synthase means a protein which has the enzymatic activity of converting geranyl-geranyl diphosphate into phytoene.

A phytoene desaturase means a protein which has the enzymatic activity of converting phytoene into phytofluene and/or phytofluene into 4-carotene (zeta-carotene).

A zeta-carotene desaturase means a protein which has the enzymatic activity of converting ζ-carotene into neurosporin and/or neurosporin into lycopene.

A crtISO protein means a protein which has the enzymatic activity of converting 7,9,7′,9′-tetra-cis-lycopene into all-trans-lycopene.

An FtsZ protein means a protein which has a cell division- and plastid division-promoting effect and displays homologies with tubulin proteins.

A MinD protein means a protein which exhibits a multifunctional role in cell division. It is a membrane-associated ATPase and may show an oscillating motion from pole to pole within the cell.

Examples of HMG-CoA reductase genes are:

A nucleic acid encoding an HMG-CoA reductase from Arabidopsis thaliana, Accession NM_{—}106299; and further HMG-CoA reductase genes from other organisms with the following Accession Numbers:

P54961, P54870, P54868, P54869, O02734, P22791, P54873, P54871, P23228, P13704, P54872, Q01581, P17425, P54874, P54839, P14891, P34135, O64966, P29057, P48019, P48020, P12683, P43256, Q9XEL8, P34136, O64967, P29058, P48022, Q41437, P12684, Q00583, Q9XHL5, Q41438, Q9YAS4, O76819, O28538, Q9Y7D2, P54960, 051628, P48021, Q03163, P00347, P14773, Q12577, Q59468, P04035, O24594, P09610, Q58116, O26662, Q01237, O01559, Q12649, O74164, O59469, P51639, O10283, O08424, P20715, P13703, P13702, Q96UG4, Q8SQZ9, O15888, Q9TUM4, P93514, Q39628, P93081, P93080, Q944T9, Q40148, Q84MM0, Q84LS3, Q9Z9N4, Q9KLM0.

Examples of (E)-4-hydroxy-3-methylbut-2-enyl-diphosphate reductase genes are:

A nucleic acid encoding an (E)-4-hydroxy-3-methylbut-2-enyl-diphosphate reductase from Arabidopsis thaliana (lytB/ISPH), ACCESSION AY168881, and further (E)-4-hydroxy-3-methylbut-2-enyl-diphosphate reductase genes from other organisms with the following Accession Numbers:

T04781, AF270978_{—}1, NP_{—}485028.1, NP_{—}442089.1, NP_{—}681832.1, ZP_{—}00110421.1, ZP_{—}00071594.1, ZP_{—}00114706.1, ISPH_SYNY3, ZP_{—}00114087.1, ZP_{—}00104269.1, AF398145_{—}1, AF398146_{—}1, AAD55762.1, AF514843_{—}1, NP_{—}622970.1, NP—348471.1, NP_{—}562001.1, NP_{—}223698.1, NP_{—}781941.1, ZP_{—}00080042.1, NP_{—}859669.1, NP_{—}214191.1, ZP_{—}00086191.1, ISPH_VIBCH, NP_{—}230334.1, NP_{—}742768.1, NP_{—}302306.1, ISPH_MYCLE, NP_{—}602581.1, ZP_{—}00026966.1, NP_{—}520563.1, NP_{—}253247.1, NP_{—}282047.1 ZP_{—}00038210.1, ZP_{—}00064913.1, CAA61555.1, ZP_{—}00125365.1, ISPH_ACICA, EAA24703.1, ZP_{—}00013067.1, ZP_{—}00029164.1, NP_{—}790656.1, NP_{—}217899.1, NP_{—}641592.1, NP_{—}636532.1, NP_{—}719076.1, NP_{—}660497.1, NP_{—}422155.1, NP_{—}715446.1, ZP_{—}00090692.1, NP_{—}759496.1, ISPH_BURPS, ZP_{—}00129657.1, NP_{—}215626.1, NP_{—}335584.1, ZP_{—}00135016.1, NP_{—}789585.1, NP_{—}787770.1, NP_{—}769647.1, ZP_{—}00043336.1, NP_{—}242248.1, ZP_{—}00008555.1, NP_{—}246603.1, ZP_{—}00030951.1, NP_{—}670994.1, NP_{—}404120.1, NP_{—}540376.1, NP_{—}733653.1, NP_{—}697503.1, NP_{—}840730.1, NP_{—}274828.1, NP_{—}796916.1, ZP_{—}00123390.1, NP_{—}824386.1, NP_{—}737689.1, ZP_{—}00021222.1, NP_{—}757521.1, NP_{—}390395.1, ZP_{—}00133322.1, CAD76178.15 NP_{—}600249.1, NP_{—}454660.1, NP_{—}712601.1, NP_{—}385018.1, NP_{—}751989.1.

Examples of 1-deoxy-D-xylose-5-phosphate synthase genes are:

A nucleic acid encoding a 1-deoxy-D-xylose-5-phosphate synthase from Lycopersicon esculentum, ACCESSION #AF143812, and further 1-deoxy-D-xylose-5-phosphate synthase genes from other organisms with the following Accession Numbers:

AF143812_{—}1, DXS_CAPAN, CAD22530.1, AF182286_{—}1, NP_{—}193291.1, T52289, AAC49368.1, AAP14353.1, D71420, DXS_ORYSA, AF443590_{—}1, BAB02345.1, CAA09804.2, NP_{—}850620.1, CAD22155.2, AAM65798.1, NP_{—}566686.1; CAD22531.1, AAC33513.1, CAC08458.1, AAG10432.1, T08140, AAP14354.1, AF428463_{—}1, ZP_{—}00010537.1, NP_{—}769291.1, AAK59424,1, NP_{—}107784.1, NP_{—}697464.1, NP_{—}540415.1, NP_{—}196699.1, NP_{—}384986.1, ZP_{—}00096461.1, ZP_{—}00013656.1, NP_{—}353769.1, BAA83576.1, ZP_{—}00005919.1, ZP_{—}00006273.1, NP_{—}420871.1, AAM48660.1, DXS_RHOCA, ZP_{—}00045608.1, ZP_{—}00031686.1, NP_{—}841218.1, ZP_{—}00022174.1, ZP_{—}00086851.1, NP_{—}742690.1, NP_{—}520342.1, ZP_{—}00082120.1, NP_{—}790545.1, ZP_{—}00125266.1, CAC17468.1, NP_{—}252733.1, ZP_{—}00092466.1, NP_{—}439591.1, NP_{—}414954.1, NP_{—}752465.1, NP_{—}622918.1, NP_{—}286162.1, NP_{—}836085.1, NP_{—}706308.1, ZP_{—}00081148.1, NP_{—}797065.1, NP_{—}213598.1, NP_{—}245469.1, ZP_{—}00075029.1, NP_{—}455016.1, NP_{—}230536.1, NP_{—}459417.1, NP_{—}274863.1, NP_{—}283402.1, NP_{—}759318,1, NP_{—}406652.1, DXS_SYNLE, DXS_SYNP7, NP_{—}440409.1, ZP_{—}00067331.1, ZP_{—}00122853.17 NP_{—}717142.1, ZP_{—}00104889.1, NP_{—}243645.1, NP_{—}681412.1, DXS SYNEL, NP_{—}637787.1, DXS-CHLTE, ZP_{—}00129863.1, NP_{—}661241.1, DXS_XANCP, NP_{—}470738.1, NP_{—}484643.1, ZP_{—}00108360.1, NP_{—}833890.1, NP_{—}846629.1, NP_{—}658213.1, NP_{—}642879.1, ZP_{—}00039479.1, ZP_{—}00060584.1, ZP_{—}00041364.1, ZP_{—}00117779.1, NP_{—}299528.1.

Examples of 1-deoxy-D-xylose-5-phosphate reductoisomerase genes are:

A nucleic acid encoding a 1-deoxy-D-xylose-5-phosphate reductoisomerase from Arabidopsis thaliana, ACCESSION #AF148852, and further 1-deoxy-D-xylose-5-phosphate reductoisomerase genes from other organisms with the following Accession Numbers:

AF148852, AY084775, AY054682, AY050802, AY045634, AY081453, AY091405, AY098952, AJ242588, AB009053, AY202991, NP_{—}201085.1, T52570, AF331705_{—}1, BAB16915.1, AF367205_{—}1, AF250235_{—}1, CAC03581.1, CAD22156.1, AF182287_{—}1, DXR_M ENPI, ZP_{—}00071219.1, NP_{—}488391.1, ZP_{—}00111307.1, DXR_SYNLE, AAP56260.1, NP_{—}681831.1, NP_{—}442113.1, ZP_{—}00115071.1, ZP_{—}00105106.1, ZP_{—}00113484.1, NP_{—}833540.1, NP_{—}657789.1, NP_{—}661031.1, DXR-BACHD, NP_{—}833080.1, NP_{—}845693.1, NP_{—}562610.1, NP_{—}623020.1, NP_{—}810915.1, NP_{—}243287.1, ZP_{—}00118743.1, NP_{—}464842.1, NP_{—}470690.1, ZP_{—}00082201.1, NP_{—}781898.1, ZP_{—}00123667.1, NP_{—}348420.1, NP_{—}604221.1, ZP_{—}00053349.1, ZP_{—}00064941.1, NP_{—}246927.1, NP_{—}389537.1, ZP_{—}00102576.15 NP_{—}519531.1, AF124757_{—}19, DXR_ZYMMO, NP_{—}713472.1, NP_{—}459225.1, NP_{—}454827.1, ZP_{—}00045738.1, NP_{—}743754.1, DXR_PSEPK, ZP_{—}00130352.1, NP_{—}702530.1, NP_{—}8417441, NP_{—}438967.1, AF514841_{—}1, NP_{—}706118.1, ZP_{—}00125845.1, NP_{—}404661.1, NP_{—}285867.1, NP_{—}240064.1, NP_{—}414715.1, ZP_{—}00094058.1, NP_{—}791365.1, ZP_{—}00012448.1, ZP_{—}00015132.1, ZP_{—}00091545.1, NP_{—}629822.1, NP_{—}771495.1, NP_{—}798691.1, NP_{—}231885.1, NP_{—}252340.1, ZP_{—}00022353.1, NP_{—}355549.1, NP_{—}420724.1., ZP_{—}00085169.1, EAA17616.1, NP_{—}273242.1, NP_{—}219574.1 NP_{—}387094.1, NP_{—}296721.1, ZP_{—}00004209.1, NP_{—}823739.1, NP_{—}282934.1, BAA77848.1, NP_{—}660577.1, NP_{—}760741.1, NP_{—}641750.1, NP_{—}636741.1, NP_{—}829309.1, NP_{—}298338.1, NP_{—}444964.1, NP_{—}717246.1, NP_{—}224545.1, ZP_{—}00038451.1, DXR_KITGR, NP_{—}778563.1.

Examples of isopentenyl-diphosphate Δ-isomerase genes are:

A nucleic acid encoding an isopentyl-diphosphate Δ-isomerase from Adonis palaestina clone ApIPI28, (ipiAa1), ACCESSION #AF188060, published by Cunningham, F. X. Jr. and Gantt, E.: Identification of multi-gene families encoding isopentenyl diphosphate isomerase in plants by heterologous complementation in Escherichia coli, Plant Cell Physiol. 41 (1), 119-123 (2000) and further isopentenyl-diphosphate Δ-isomerase genes from other organisms with the following Accession Numbers:

Q38929, O48964, Q39472, Q13907, O35586, P58044, O42641, O35760,Q10132, P15496, Q9YB30, Q8YNH4, Q42553, O27997, P50740, O51627, O48965, Q8KFR5, Q39471, Q39664, Q9RVE2, Q01335, Q9HHE4, Q9BXS1, Q9KWF6, Q9CIF5, Q88WB6, Q92BX2, Q8Y7A5, Q8TT35 Q9KK75, Q8NN99, Q8XD58, Q7FE75, Q46822, Q9HP40, P72002, P26173, Q9Z5D3, Q8Z3X9, Q8ZM82, Q9X7Q6, O13504, Q9HFW8, Q8NJL9, Q9UUQ1, Q9NH02, Q9M6K9, Q9M6K5, Q9FXR6, O081691, Q9S7C4, Q8S3L8, Q9M592, Q9M6K3, Q9M6K7, Q9FV48, Q9LLB6, Q9AVJ1, Q9AVG8, Q9M6K6, Q9AVJ5, Q9M6K2, Q9AYS5, Q9M6K8, Q9AVG7, Q8S3L7, Q8W250, Q94IE1, Q9AVI8, Q9AYS6, Q9SAY0, Q9M6K4, Q8GVZ0, Q84RZ8, Q8KZ12, Q8KZ66, Q8FND7, Q88QC9, Q8BFZ6, BAC26382, CAD94476.

Examples of geranyl-diphosphate synthase genes are:

A nucleic acid encoding a geranyl-diphosphate synthase from Arabidopsis thaliana, ACCESSION #Y17376, Bouvier, F., Suire, C., d'Harlingue, A., Backhaus, R. A. and Camara, B., Molecular cloning of geranyl diphosphate synthase and compartmentation of monoterpene synthesis in plant cells, Plant J. 24 (2), 241-252 (2000) and further geranyl-diphosphate synthase genes from other organisms with the following Accession Numbers:

Q9FT89, Q8LKJ2, Q9FSW8, Q8LKJ3, Q9SBR3, Q9SBR4, Q9FET8, Q7LKJ1, Q84LG1, Q9JK86

Examples of farnesyl-diphosphate synthase genes are:

A nucleic acid encoding a farnesyl-diphosphate synthase from Arabidopsis thaliana (FPS1), ACCESSION #U80605, published by Cunillera, N., Arro, M., Delourme, D., Karst, F., Boronat, A. and Ferrer, A.: Arabidopsis thaliana contains two differentially expressed farnesyl-diphosphate synthase genes, J. Biol. Chem. 271 (13), 7774-7780 (1996), and further farnesyl-diphosphate synthase genes from other organisms with the following Accession Numbers:

P53799, P37268, Q02769, Q09152, P49351, O24241, Q43315, P49352, O24242, P49350, P08836, P14324, P49349, P08524, O66952, Q082911 P54383, Q45220, P57537, Q8K9A0, P22939, P45204, O66126, P55539, Q9SWH9, Q9AVI7, Q9FRX2, Q9AYS7, Q94IE8, Q9FXR9, Q9ZWF6, Q9FXR8, Q9AR37, O50009, Q94IE9, Q8RVK7, Q8RVQ7, O04882, Q93RA8, Q93RB0, Q93KB4, Q93RB5, Q93RB3, O93RB1, Q93RB2, Q920E5.

Examples of geranyl-geranyl-diphosphate synthase genes are:

A nucleic acid encoding a geranyl-geranyl-diphosphate synthase from Sinapis alba, ACCESSION #X98795, published by Bonk, M., Hoffmann, B., Von Lintig, J., Schledz, M., Al-Babili, S., Hobeika, E., Kleinig, H. and Beyer, P.: Chloroplast import of four carotenoid biosynthetic enzymes in vitro reveals differential fates prior to membrane binding and oligomeric assembly, Eur. J. Biochem. 247 (3), 942-950 (1997), and further geranyl-geranyl-diphosphate synthase genes from other organisms with the following Accession Numbers:

P22873, P34802, P56966, P80042, Q42698, Q92236, O95749, Q9WTN0, Q50727, P24322, P39464, Q9FXR3, Q9AYN2, Q9FXR2, Q9AVG6, Q9FRW4, Q9SXZ5, Q9AVJ7, Q9AYN1, Q9AVJ4, Q9FXR7, Q8LSC5, Q9AVJ6, Q8LSC4, Q9AVJ3, Q9SSU0, Q9SXZ6, Q9SST9, Q9AVJ0, Q9AV19, Q9FRW3, Q9FXR5, Q94IF0, Q9FRX1, Q9K567, Q93RA9, Q93QX8, CAD95619, EAA31459.

Examples of phytoene synthase genes are:

A nucleic acid encoding a phytoene synthase from Erwinia uredovora, ACCESSION #D90087; published by Misawa, N., Nakagawa, M., Kobayashi, K., Yamano, S., Izawa, Y., Nakamura, K. and Harashima, K., Elucidation of the Erwinia uredovora carotenoid biosynthetic pathway by functional analysis of gene products expressed in Escherichia coli; J. Bacteriol. 172 (12), 6704-6712 (1990), and further phytoene synthase genes from other organisms with the following Accession Numbers:

CAB39693, BAC69364, AAF10440, CAA45350, BMA20384, AAM72615, BAC09112, CAA48922, P_{—}001091, CAB84588, AAF41518, CAA48155, AAD38051, AAF33237, AAG10427, AAA34187, BAB73532, CAC19567, AAM62787, CAA55391, AAB65697, AAM45379, CAC27383, AAA32836, AAK07735, BAA84763, P_{—}000205, AAB60314, P_{—}001163, P_{—}000718, AAB71428, AAA34153, AAK07734, CAA42969, CAD76176, CAA68575, P_{—}000130, P_{—}001142, CAA47625, CAA85775, BAC14416, CAA79957; BAC76563, P_{—}000242, P_{—}000551, AAL02001, AAK15621, CAB94795, AAA91951, P_{—}000448.

Examples of phytoene desaturase genes are:

A nucleic acid encoding a phytoene desaturase from Erwinia uredovora, ACCESSION #D90087; published by Misawa, N., Nakagawa, M., Kobayashi, K., Yamano, S., Izawa, Y., Nakamura, K. and Harashima, K.: Elucidation of the Erwinia uredovora carotenoid biosynthetic pathway by functional analysis of gene products expressed in Escherichia coli; J. Bacteriol. 172 (12), 6704-6712 (1990), and further phytoene desaturase genes from other organisms with the following Accession Numbers:

AAL15300, A39597, CAA42573, AAK51545, BAB08179, CAA48195, BAB82461, AAK92625, CAA55392, AAG10426, AAD02489, AA024235, AAC12846, AAA99519, AAL38046, CAA60479, CAA75094, ZP_{—}001041. ZP_{—}001163, CAA39004, CAA44452, ZP_{—}001142, ZP_{—}000718, BAB82462, AAM45380, CAB56040, ZP_{—}001091, BAC09113, AAP79175, AAL80005, AAM72642, AAM72043, ZP_{—}000745, ZP_{—}001141, BAC07889, CAD55814, ZP_{—}001041, CAD27442, CAE00192, ZP_{—}001163, ZP_{—}000197, BAA18400, AAG10425, ZP_{—}001119, AAF13698, 2121278A, AAB35386, AAD02462, BAB68552, CAC85667, MK51557, CAA12062, AAG51402, AAM63349, AAF85796, BAB74081, AAA91161, CAB56041, AAC48983, AAG14399, CAB65434, BAB73487, ZP_{—}001117, ZP_{—}000448, CAB39695, CAD76175, BAC69363, BAA17934, ZP_{—}000171, AAF65586, ZP_{—}000748, BAC07074, ZP_{—}001133, CAA64853, BAB74484, ZP_{—}001156, AAF23289, AAG28703, MAP09348, AAM71569, BAB69140, ZP_{—}000130, AAF41516, AAG18866, CAD959407 NP_{—}656310, AAG10645, ZP_{—}000276, ZP_{—}000192, ZP_{—}000186, AAM94364, EAA31371, ZP_{—}000612, BAC75676, AAF65582.

Examples of zeta-carotene desaturase genes are:

A nucleic acid encoding a zeta-carotene desaturase from Narcissus pseudonarcissus, ACCESSION #AJ224683, published by Al-Babili, S., Oelschlegel, J. and Beyer, P.: A cDNA encoding for beta carotene desaturase (Accession No. AJ224683) from Narcissus pseudonarcissus L. (PGR98-103), Plant Physiol. 117, 719-719 (1998), and further zeta-carotene desaturase genes from other organisms with the following Accession Numbers: Q9R6X4, Q38893, Q9SMJ3, Q9SE20, Q9ZTP4, O49901, P74306, Q9FV46, Q9RCT2, ZDS_NARPS, BAB68552.1, CAC85667.1, AF372617_{—}1, ZDS_TARER, CAD55814.1, CAD27442.1, 2121278A, ZDS_CAPAN, ZDS_LYCES, NP_{—}187138.1, AAM63349.1, ZDS_ARATH, AAA91161,1, ZDS_MAIZE, AAG14399.1, NP_{—}441720.1, NP_{—}486422.1, ZP_{—}00111920.1, CAB56041.1, ZP_{—}00074512.1, ZP_{—}00116357.1, NP_{—}681127.1, ZP_{—}00114185.1, ZP_{—}00104126.1, CAB65434.1, NP_{—}662300.1.

Examples of crtISO genes are:

A nucleic acid encoding a crtISO from Lycopersicon esculentum; ACCESSION #AF416727, published by isaacson, T., Ronen, G., Zamir, D. and Hirschberg, J.: Cloning of tangerine from tomato reveals a carotenoid isomerase essential for the production of beta-carotene and xanthophylis in plants; Plant Cell 14 (2), 333-342 (2002), and further crtISO genes from other organisms with the following Accession Numbers:

AAM53952

Examples of FtsZ genes are:

A nucleic acid encoding an FtsZ from Tagetes erecta, ACCESSION #AF251346, published by Moehs, C. P., Tian, L., Osteryoung, K. W. and Dellapenna, D.: Analysis of carotenoid biosynthetic gene expression during marigold petal development Plant Mol. Biol. 45 (3), 281-293 (2001), and further FtsZ genes from other organisms with the following Accession Numbers:

CAB89286.1, AF205858_{—}1, NP_{—}200339.1, CAB89287.1, CAB41987.1, AAA82068.1, T06774, AF383876_{—}1, BAC57986.1, CAD22047.1, BAB91150.1, ZP_{—}00072546.1, NP_{—}440816.1, T51092, NP_{—}683172.1, BAA85116.1, NP_{—}487898.1, JC4289, BAA82871,1, NP_{—}781763.1, BAC57987.1, ZP_{—}00111461.1, T51088, NP_{—}190843.1, ZP_{—}00060035.1, NP_{—}846285.1, AAL07180.1, NP_{—}243424.1, NP_{—}833626.1, AAN04561.1, AAN04557.1, CAD22048.1, T51089, NP_{—}692394.1, NP_{—}623237.1, NP_{—}565839.1, T51090, CAA07676.1, NP_{—}113397.1, T51087, CAC44257.1, E84778, ZP_{—}00105267.1, BAA82091.1, ZP_{—}00112790.1, BAA96782.1, NP_{—}348319.1, NP_{—}471472.1, ZP_{—}00115870.1, NP_{—}465556.1, NP_{—}389412.1, BAA82090.1, NP_{—}562681.1, AAM22891.1, NP_{—}371710.1, NP_{—}764416.1, CAB95028.1, FTSZ_STRGR, AF120117_{—}1, NP_{—}827300.1, JE0282, NP_{—}626341.1, AAC45639.1, NP_{—}785689.1, NP_{—}336679.1, NP_{—}738660.17 ZP_{—}00057764.1, AAC32265.1, NP_{—}814733.1, FTSZ_MYCKA, NP_{—}216666.1, CAA75616.1, NP_{—}301700.1, NP_{—}601357.1, ZP_{—}00046269.1, CAA70158.1, ZP_{—}00037834.1, NP_{—}268026.1, FTSZ_ENTHR, NP_{—}787643.1, NP_{—}346105.1, AAC32264.1, JC5548, AAC95440.1, NP_{—}710793.1 NP_{—}687509.1, NP_{—}269594.1, AAC32266.1, NP_{—}720988.1, NP_{—}657875.17 ZP_{—}00094865.1, ZP_{—}00080499.1, ZP_{—}00043589.1, JC7087, NP_{—}660559.1, AAC46069.1, AF179611_{—}14, AAC44223.1, NP_{—}404201.1.

Examples of MinD genes are:

A nucleic acid encoding an MinD from Tagetes erecta, ACCESSION #AF251019, published by Moehs, C. P., Tian, L., Osteryoung, K. W. and Dellapenna, D.: Analysis of carotenoid biosynthetic gene expression during marigold petal development; Plant Mol. Biol. 45 (3), 281-293 (2001), and further MinD genes with the following Accession Numbers:

NP_{—}197790.1, BAA90628.1, NP_{—}038435.1, NP_{—}045875.1, AAN33031.1, NP_{—}050910.1, CAB53105.1, NP_{—}050687.1, NP_{—}682807.1, NP_{—}487496.1, ZP_{—}00111708.1, ZP_{—}00071109.1, NP_{—}442592.1, NP_{—}603083.1, NP_{—}782631.1, ZP_{—}00097367.1, ZP_{—}00104319.1, NP_{—}294476.1, NP_{—}622555.1, NP_{—}563054.1, NP_{—}347881.1, ZP_{—}00113908.1, NP_{—}834154.1, NP_{—}658480.1, ZP_{—}00059858.1, NP_{—}470915.1, NP_{—}243893.1, NP_{—}465069.1, ZP_{—}00116155.1, NP_{—}390677.1, NP_{—}692970.1, NP_{—}298610.1, NP_{—}207129.1, ZP_{—}00038874.1, NP_{—}778791.1, NP_{—}223033.1, NP_{—}641561.1, NP_{—}636499.1, ZP_{—}00088714.1, NP_{—}213595.1, NP_{—}743889.1, NP_{—}231594.1, ZP_{—}00085067.1, NP_{—}797252.1, ZP_{—}00136593.1, NP_{—}251934.1, NP_{—}405629.1, NP_{—}759144.1, ZP_{—}00102939.1, NP_{—}793645.1, NP_{—}699517.1, NP_{—}460771.1, NP_{—}860754.1, NP_{—}456322.1, NP_{—}718163.1, NP_{—}229666.1, NP_{—}357356.1, NP_{—}541904.1, NP_{—}287414.1, NP_{—}660660.1, ZP_{—}00128273.1, NP_{—}103411.1, NP_{—}785789.1, NP_{—}715361.1, AF149810_{—}1, NP_{—}841854.1, NP_{—}437893.1, ZP_{—}00022726.1, EAA24844.1, ZP_{—}00029547.1, NP_{—}521484.1, NP_{—}240148.1, NP_{—}770852.1, AF345908_{—}2, NP_{—}777923.1, ZP_{—}00048879.1, NP_{—}579340.1, NP_{—}143455.1, NP_{—}126254.1 NP_{—}142573.1, NP_{—}613505.1, NP_{—}127112.1, NP_{—}712786.1, NP_{—}578214.1, NP_{—}069530.1, NP_{—}247526.1, AAA85593.1, NP_{—}212403.1, NP_{—}782258.1, ZP_{—}00058694.1, NP_{—}247137.1, NP_{—}219149.1, NP_{—}276946.1, NP_{—}614522.1, ZP_{—}00019288.1, CAD78330.1.

The invention further relates to a genetically modified plant of the genus Tagetes, where the genetic modification leads to an increase or causing of the expression rate of at least one gene compared with the wild type and is caused by regulation of the expression of this gene in the plant by the promoters of the invention.

In a preferred embodiment of the genetically modified plants of the invention of the genus Tagetes, the regulation of the expression of genes in the plant by the promoters of the invention is achieved by

a) introducing one or more promoters of the invention into the genome of the plant so that expression of one or more endogenous genes takes place under the control of the introduced prorhoters of the invention, or

b) introducing one or more genes into the genome of the plant, so that expression of one or more of the introduced genes takes place under the control of the endogenous promoters of the invention, or

c) introducing one or more nucleic acid constructs comprising at least one promoter of the invention and, functionally linked, one or more genes to be expressed into the plant.

In a preferred embodiment, according to feature c) one or more nucleic acid constructs comprising at least one promoter of the invention and, functionally linked, one or more genes to be expressed are introduced into the plant. Integration of the nucleic acid constructs in the plant of the genus Tagetes can take place in this case intrachromosomally or extrachromosomally.

Preferred promoters of the invention and preferred genes to be expressed (effect genes) are described above.

The production of the genetically modified plants of the genus Tagetes with increased or caused expression rate of an effect gene is described by way of example below.

The transformation can in the case of combinations of genetic modifications take place singly or by multiple constructs.

The transgenic plants are preferably produced by transformation of the initial plants with a nucleic acid construct which comprises at least one of the promoters of the invention described above which are functionally linked to an effect gene to be expressed and, if appropriate, further regulatory signals.

These nucleic acid constructs in which the promoters of the invention and effect genes are functionally linked are also called expression cassettes hereinafter.

The expression cassettes may comprise further regulatory signals, that is regulatory nucleic acid sequences which control the expression of the effect genes in the host cell. In a preferred embodiment, an expression cassette comprises at least one promoter of the invention upstream, i.e. at the 5′ end of the coding sequence, and a polyadenylation signal and, if appropriate, further regulatory elements which are operatively linked to the coding sequence, lying inbetween, of the effect gene for at least one of the genes described above, downstream, i.e. at the 3′ end.

An operative linkage means the sequential arrangement of promoter, coding sequence, terminator and, if appropriate, further regulatory elements in such a way that each of the regulatory elements can complete its function as intended in the expression of the coding sequence.

The preferred nucleic acid constructs, expression cassettes and vectors for plants and processes for producing transgenic plants, and the transgenic plants of the genus Tagetes themselves, are described by way of example below.

The sequences which are preferred for the operative linkage, but are not restricted thereto, are targeting sequences to ensure subcellular localization in the apoplast, in the vacuole, in plastids, in the mitochondrion, in the endoplasmic reticulum (ER), in the cell nucleus, in elaioplasts or other compartments and translation enhancers such as the 5′-leader sequence from tobacco mosaic virus (Gallie et al., Nucl. Acids Res. 15 (1987), 8693-8711).

An expression cassette is preferably produced by fusing at least one promoter of the invention to at least one gene, preferably to one of the effect genes described above, and preferably a nucleic acid which is inserted after the promoter and nucleic acid sequence and codes for a plastid-specific transit peptide, and in front of a polyadenylation signal by conventional techniques of recombination and cloning as described for example in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

The nucleic acids encoding a plastid transit peptide which are preferably inserted ensure the localization in plastids and especially in chromoplasts.

It is also possible to use expression cassettes whose nucleic acid sequence codes for an effect gene product fusion protein, in which case part of the fusion protein is a transit peptide which controls translocation of the polypeptide. Preference is given to transit peptides which are specific for chromoplasts and which, after translocation of the effect genes into the chromoplasts, are eliminated enzymatically from the effect gene product part.

Particular preference is given to the transit peptide which is derived from the Nicotiana tabacum plastid transketolase or from another transit peptide (e.g. the transit peptide of the small subunit of Rubisco (rbcS) or of the ferredoxin NADP oxidoreductase and of the isopentenyi-pyrophosphate isomerase 2 or its functional equivalent.

Further examples of a plastid transit peptide are the transit peptide of the plastid isopentenyl-pyrophosphate isomerase-2 (IPP-2) from Arabidsopsis thaliana and the transit peptide of the small subunit of ribulose-bisphosphate carboxylase (rbcS) from pea (Guerineau, F, Woolston, S, Brooks, L, Mullineaux, P (1988) An expression cassette for targeting foreign proteins into the chloroplasts. Nucl. Acids Res. 16: 11380).

The nucleic acids of the invention can be produced by synthesis or be obtained naturally or comprise a mixture of synthetic and natural nucleic acid constituents, and consist of various heterologous gene segments from different organisms.

Preference is given, as described above, to synthetic nucleotide sequences with codons preferred by plants. These codons preferred by plants can be determined from codons with the highest protein frequency which are expressed in most of the plant species of interest.

It is possible in the preparation of an expression cassette to manipulate various DNA fragments in order to obtain a nucleotide sequence which expediently reads in the correct direction and which is equipped with a correct reading frame. Adaptors or linkers can be attached to the fragments to join the DNA fragments together.

It is expediently possible for the promoter and terminator regions to be provided, in the direction of transcription, with a linker or polylinker comprising one or more restriction sites for inserting this sequence. Ordinarily, the linker has 1 to 10, in most cases 1 to 8, preferably 2 to 6, restriction sites. In general, the linker within the regulatory regions has a size of less than 100 bp, frequently less than 60 bp, but at least 5 bp. The promoter can be both native, or homologous, and foreign, or heterologous, with regard to the host plant. The expression cassette preferably comprises, in the 5′-3′ direction of transcription, the promoter, a coding nucleic acid sequence or a nucleic acid construct and a region for termination of transcription. Different termination regions can be mutually exchanged as desired.

Examples of a terminator are the 358 terminator (Guerineau et al. (1988) Nucl Acids Res. 16: 11380), the nos terminator (Depicker A, Stachel S, Dhaese P, Zambryski P, Goodman H M. Nopaline synthase: transcript mapping and DNA sequence. J Mol Appl Genet. 1982: 1(6):561-73) or the ocs terminator (Gielen, J, de Beuckeleer, M, Seurinck, J, Debroek, H, de Greve, H, Lemmers, M, van Montagu, M, Schell, J (1984) The complete sequence of the TL-DNA of the Agrobacterium tumefaciens plasmid pTiAch5. EMBO J, 3: 835-846).

It is moreover possible to employ manipulations which provide suitable restriction cleavage sites or which remove surplus DNA or restriction cleavage sites. Where insertions, deletions or substitutions such as, for example, transitions and tranversions are suitable, it is possible to use in vitro mutagenesis, “primer repair”, restriction or ligation.

In the case of suitable manipulations such as, for example, restriction, chewing back or filling in of overhangs for blunt ends, it is possible to provide complementary ends of the fragments for the ligation.

Preferred polyadenylation signals are plant polyadenylation signals, preferably those substantially corresponding to T-DNA polyadenylation signals from Agrobacterium tumefaciens, especially of gene 3 of the T-DNA (octopine synthase) of the Ti plasmid pTiACH5 (Gielen et al., EMBO J. 3,(1984), 835 ft) or functional equivalents.

The transfer of foreign genes into the genome of a plant is referred to as transformation.

It is possible to use for this purpose methods known per se for the transformation and regeneration of plants from plant tissues or plant cells for transient or stable transformation.

Suitable methods for transforming plants are protoplast transformation by polyethylene glycol-induced DNA uptake, the biolistic method with the gene gun—the so-called particle bombardment method, electroporation, incubation of dry embryos in DNA-containing solution, microinjection, and the gene transfer mediated by Agrobacterium as described above. Said methods are described for example in B. Jenes et al., Techniques for Gene Transfer, in: Transgenic Plants, Vol. 1, Engineering and Utilization, edited by S. D. Kung and R. Wu, Academic Press (1993), 128-143 and in Potrykus, Annu. Rev. Plant Physiol. Plant Molec. Biol. 42 (1991), 205-225).

The construct to be expressed is preferably cloned into a vector which is suitable for transformation of Agrobacterium tumefaciens, for example pBin19 (Bevan et al., Nucl. Acids Res. 12 (1984), 8711) or particularly preferably pSUN2, pSUN3, pSUN4 or pSUN5 (WO 02/00900).

Agrobacteria transformed with an expression plasmid can be used in a known manner for the transformation of plants, e.g. by bathing wounded leaves, pieces of leaf or cotyledons in a solution of Agrobacteria and subsequently cultivating in suitable media.

For the preferred production of genetically modified plants, also referred to as transgenic plants hereinafter, the fused expression cassette is cloned into a vector, for example pBin19 or, in particular, pSUN5 and pSUN3, which is suitable to be transformed into Agrobacterium tumefaciens. Agrobacteria transformed with such a vector can then be used in a known manner for the transformation of plants, in particular of crop plants, by for example bathing wounded leaves, pieces of leaf or cotyledons in a solution of Agrobacteria and subsequently cultivating in suitable media.

The transformation of plants by Agrobacteria is disclosed inter alia in F. F. White, Vectors for Gene Transfer in Higher Plants; in Transgenic Plants, Vol. 1, Engineering and Utilization, edited by S. D. Kung and R. Wu, Academic Press, 1993, pp. 15-38. Transgenic plants comprising one or more genes integrated into the expression cassette can be regenerated in a known manner from the transformed cells of the wounded leaves, pieces of leaf or cotyledons.

To transform a host plant with one or more effect genes of the invention, an expression cassette is incorporated as insertion into a recombinant vector whose vector DNA comprises additional functional regulatory signals, for example sequences for replication or integration. Suitable vectors are described inter alia in “Methods in Plant Molecular Biology and Biotechnology” (CRC Press), chapters 6/7, pp. 71-119 (1993).

It is possible by using the recombination and cloning techniques cited above to clone the expression cassettes into suitable vectors which make their replication possible, for example in E. coli. Suitable cloning vectors are inter alia pJIT117 (Guerineau et al. (1988) Nucl. Acids Res. 16: 11380), pBR332, pUC series, M13mp series and pACYCI84. Binary vectors able to replicate both in E. coli and in Agrobacteria are particularly suitable.

Preferred promoters of the invention and preferred effect genes are described above.

Particularly preferred effect genes are those selected from the group of nucleic acids encoding a ketolase, nucleic acids encoding a β-hydroxylase, nucleic acids encoding a β-cyclase, nucleic acids encoding an ε-cyclase, nucleic acids encoding an epoxidase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl-diphosphate Δ-isomerase, nucleic acids encoding a geranyl-diphosphate synthase, nucleic acids encoding a farnesyl-diphosphate synthase, nucleic acids encoding a geranyl-geranyl-diphosphate synthase, nucleic acids encoding a phytoene synthase, nucleic acids encoding a phytoene desaturase, nucleic acids encoding a prephytoene synthase, nucleic acids encoding a zeta-carotene desaturase, nucleic acids encoding a crtISO protein, nucleic acids encoding an FtsZ protein and nucleic acids encoding an MinD protein.

Preferred genetically modified plants of the genus Tagetes are marigold, Tagetes erecta, Tagetes patula, Tagetes lucida, Tagetes pringlei, Tagetes palmeri, Tagetes minuta or Tagetes campanulata.

It is possible with the aid of the processes of the invention described above to regulate through the promoters of the invention the metabolic pathways to specific biosynthetic products in the genetically modified plants of the invention described above of the genus Tagetes.

For this purpose, for example, metabolic pathways which lead to a specific biosynthetic product are enhanced by causing or increasing the transcription rate or expression rate of genes of this biosynthetic pathway through the increased amount of protein leading to an increased total activity of these proteins of the desired biosynthetic pathway and thus to an enhanced metabolic flux toward the desired biosynthetic product.

It is necessary, depending on the desired biosynthetic product, to increase or reduce the transcription rate or expression rate of various genes. It is ordinarily advantageous to alter the transcription rate or expression rate of a plurality of genes, i.e. to increase the transcription rate or expression rate of a combination of genes and/or to reduce the transcription rate or expression rate of a combination of genes.

In the genetically modified plants of the invention, at least one increased or caused expression rate of a gene is attributable to a promoter of the invention.

Further, additionally altered, i.e. additionally increased or additionally reduced, expression rates of further genes in genetically modified plants may, but need not, be derived from the promoters of the invention.

The invention therefore relates to a process for producing biosynthetic products by cultivating genetically modified plants of the invention of the genus Tagetes.

The invention relates in particular to a process for producing carotenoids by cultivating genetically modified plants of the invention of the genus Tagetes, wherein the genes to be expressed are selected from the group of nucleic acids encoding a ketolase, nucleic acids encoding a β-hydroxylase, nucleic acids encoding a β-cyclase, nucleic acids encoding an ε-cyclase, nucleic acids encoding an epoxidase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl-diphosphate Δ-isomerase, nucleic acids encoding a geranyl-diphosphate synthase, nucleic acids encoding a farnesyl-diphosphate synthase, nucleic acids encoding a geranyl-geranyl-diphosphate synthase, nucleic acids encoding a phytoene synthase, nucleic acids encoding a phytoene desaturase, nucleic acids encoding a prephytoene synthase, nucleic acids encoding a zeta-carotene desaturase, nucleic acids encoding a crtISO protein, nucleic acids encoding an FtsZ protein and nucleic acids encoding an MinD protein.

The carotenoids are preferably selected from the group of phytoene, phytofluene, lycopene, lutein, beta-carotene, zeaxanthin, astaxanthin, canthaxanthin, echinenone, 3-hydroxyechinenone, 3′-hydroxyechinenone, adonirubin, violaxanthin and adonixanthin.

The invention further relates in particular to a process for producing ketocarotenoids by cultivating genetically modified plants of the invention of the genus Tagetes, wherein the genes to be expressed are selected from the group of nucleic acids encoding a ketolase, nucleic acids encoding a β-hydroxylase, nucleic acids encoding a β-cyclase, nucleic acids encoding an ε-cyclase, nucleic acids encoding an epoxidase, nucleic acids encoding an HMG-CoA reductase, nucleic acids encoding an (E)-4-hydroxy-3-methylbut-2-enyl-diphosphate reductase, nucleic acids encoding a 1-deoxy-D-xylose-5-phosphate synthase, nucleic acids encoding a 1-deoxy-D-xylose-5-phosphate reductoisomerase, nucleic acids encoding an isopentenyl-diphosphate Δ-isomerase, nucleic acids encoding a geranyl-diphosphate synthase, nucleic acids encoding a farnesyl-diphosphate synthase, nucleic acids encoding a geranyl-geranyl-diphosphate synthase, nucleic acids encoding a phytoene synthase, nucleic acids encoding a phytoene desaturase, nucleic acids encoding a prephytoene synthase, nucleic acids encoding a zeta-carotene desaturase, nucleic acids encoding a crtISO protein, nucleic acids encoding an FtsZ protein and nucleic acids encoding an MinD protein.

The ketocarotenoids are preferably selected from the group of astaxanthin, canthaxanthin, echinenone, 3-hydroxyechinenone, 3′-hydroxyechinenone, adonirubin and adonixanthin.

In the process of the invention for producing biosynthetic products, especially carotenoids, preferably ketocarotenoids, the step of cultivating the genetically modified plants is preferably followed by a harvesting of the plants and an isolation of the biosynthetic products, especially carotenoids, preferably ketocarotenoids, from the plants, preferably from the petals of the plants.

The genetically modified plants of the genus Tagetes are grown on nutrient media in a manner known per se and appropriately harvested.

Ketocarotenoids are isolated from the harvested petals for example in a manner known per se, for example by drying and subsequent extraction and, if appropriate, further chemical or physical purification processes such as, for example, precipitation methods, crystallography, thermal separation methods such as rectification methods or physical separation methods such as, for example, chromatography. Ketocarotenoids are isolated from the petals for example preferably by organic solvents such as acetone, hexane, heptane, ether or tert-butyl methyl ether.

Further methods for isolating ketocarotenoids, especially from petals, are described for example in Egger and Kleinig (Phytochemistry (1967) 6, 437-440) and Egger (Phytochemistry (1965) 4, 609-618).

A particularly preferred ketocarotenoid is astaxanthin.

The ketocarotenoids result in the process of the invention in the form of their mono- or diesters with fatty acids in the petals. Some fatty acids which have been detected are for example myristic acid, palmitic acid, stearic acid, oleic acid, linolenic acid and lauric acid (Kamata and Simpson (1987) Comp. Biochem. Physiol. Vol. 86B(3), 587-591).

Genetically modified plants or parts of plants of the invention which can be consumed by humans and animals, such as in particular petals, with an increased content of biosynthetic products, in particular carotenoids, in particular ketocarotenoids, in particular astaxanthin, can also be used for example directly or after processing known per se as human or animal food or as supplement to animal and human foods.

The genetically modified plants can moreover be used to produce extracts containing biosynthetic products, in particular carotenoids, in particular ketocarotenoids, in particular astaxanthin, and/or for producing supplements for animal and human foods, and cosmetics and pharmaceuticals.

The genetically modified plants of the genus Tagetes have by comparison with the wild type an increased content of the desired biosynthetic products, in particular carotenoids, in particular ketocarotenoids, in particular astaxanthin.

An increased content also means in this case a caused content of ketocarotenoids, or astaxanthin.

Experimental Section

### Example 1

Amplification of a DNA which corresponds to the complete primary sequence of the LEPAP2 promoter from Lycopersicon esculentum.

The DNA sequence which codes for the PAP2 promoter from Lycopersicon esculentum was isolated by thermal-interlaced (TAIL) PCR (Liu et al. 1995 Plant J 457-463, Tsugeki et al. 1996 Plant J 479489) from Lycopersicon esculentum var. Moneymaker.

The TAIL-PCR method can be used to isolate unknown DNA fragments which flank a known DNA sequence. In this example, the sequence which is located upstream of the 5′ end of the genomic sequence which corresponds to the tomato EST clone EST554295 (SEQ ID No. 01) (Accession B1934406, tomato flower, anthesis Lycopersicon esculentum cDNA clone cTOD19B12 5′ end, mRNA sequence) was isolated. Three different primers with antisense orientation to the tomato EST clone EST554295 were derived. These primers were employed singly in consecutive PCR reactions with any degenerate primer.

The TAIL-PCR was carried out in accordance with an adapted protocol of the method of Liu et al. (1995 Plant J 8(3):457-463) and Tsugeki et al. (1996 Plant J 10(3):479-489). The following mastermix (data per reaction mixture) is employed for a first PCR reaction.

11 μl of sterile H₂O (double distilled)

2 μl of primer stock solution of the specific primer LEPAP2-TAIL-1 (5 mM) (SEQ ID No. 02)

3 μl of AD1 primer stock solution (20 mM) (SEQ ID No. 05)

2 μl of 10× PCR buffer (TAKARA)

2 μl of 10× dNTP mix (2 mM)

0.2 μl of Taq polymerase (TAKARA)

20.2 μl of this mastermix are pipetted into 1 μl of a preparation of genomic DNA from Lycopersicon esculentrum var. Moneymaker (preparation according to Galbiati M et al. (2000) Funct Integr Genomics: 25-34) in a PCR vessel and thoroughly mixed by pipetting. The primary PCR reaction is carried out under the following conditions:
94° C. for 1 min
four cycles at 94° C. for 10 s, 62° C. for 1 min and 72° C. for 150 s
94° C. for 10 s, 25° C. for 3 min, 0.20 C/s to 72° C. and 72° C. for 150 s
fourteen cycles at 94° C. for 10 s, 69° C. for 1 min, 72° C. for 150 s, 94° C. for 10 s, 68° C. for 1 min, 72° C. for 150 s, 9420 C. for 10 s, 44° C. for 1 min and 72° C. for 150 s
72° C. for 5 min, then 4° C. until used further.

The product of the PCR reaction is diluted 1:50 and 1 μl of each diluted sample is used for a second PCR reaction (secondary PCR). The following mastermix (data per reaction mixture) is employed for this:

11 μl of sterile H₂O (double distilled)

2 μl of primer stock solution of the specific primer LEPAP2-TAIL-2 (5 mM) (SEQ ID No. 03)

2 μl of AD1 primer stock solution (20 mM) (SEQ ID No. 05)

2 μl of 10× PCR buffer (TAKARA)

2 μl of 10× dNTP mix (2 mM)

0.2 μl of Taq polymerase (TAKARA)

19.2 μl portions of the second mastermix are added to 1 μl portions of the primary PCR product diluted 1:50, and the secondary PCR is carried out under the following conditions:
11 cycles at 94° C. for 10 s, 64° C. for 1 min, 72° C. for 150 s, 94° C. for 10 s, 64° C. for 1 min, 72° C. for 150 s, 94° C. for 10 s, 44° C. for 1 min, 72° C. for 150 s,
72° C. for 5 min, then 4° C. until used further.

The PCR product of the secondary PCR reaction is diluted 1:10 and 1 μl of each diluted sample is used for a third PCR reaction (tertiary PCR). The following mastermix (data per reaction mixture) is employed for this:

18 μl of sterile H₂O (double distilled)

3 μl of 10× PCR buffer (TAKARA)

3 μl of 10× dNTP mix (2 mM)

2 μl of primer stock solution of the specific primer LEPAP2-TAIL3 (5 mM) (SEQ ID No. 04)

3 μl of AD1 primer stock solution (20 mM) (SEQ ID No. 05)

0.5 μl of Taq polymerase (TAKARA)

29.5 μl portions of this mastermix are added to 1 μl portions of the secondary PCR product diluted 1:10, and the tertiary PCR is carried out under the following conditions:

19 cycles at 94° C. for 15 s, 44° C. for 1 min, 72° C. for 150 s, 72° C. for 5 min, then 4° C. until used further.

The PCR amplification described above with SEQ ID No. 04 and SEQ ID No. 05 resulted in a 997 bp fragment which consists of the promoter and a short segment at the 5′ terminus of the coding DNA sequence of the LEPAP2 gene from Lycopersicon esculentum (Seq ID No. 6). This fragment was cloned in the PCR cloning vector pCR 2.1 (Invitrogen) using standard methods, and the clone pCRLEPAP2TAIL was obtained.

The primers LEPAP2-F (SEQ ID NO. 07) and LEPAP2-R (SEQ ID NO. 08) were derived on the basis of the sequence of the insert pCRLEPAP2TAIL in order to amplify the correct sequence of the LEPAP2 promoter of Lycopersicon esculentum (without the mutations introduced owing to multiple rounds of TAIL-PCR). For this purpose, these primers were employed in a specific PCR reaction with genomic DNA from Lycopersicon esculenturm as template.

The PCR conditions were as follows:

The PCR for amplifying the LEPAP2 promoter from Lycopersicon esculentum took place in a 50 μl reaction mixture which comprised:

2 μl of a Lycopersicon esculentum DNA

5 μl of 10× dNTP mix (2 mM)

5 μl of LEPAP2-F (5 mM) (SEQ ID NO. 07)

5 μl of LEPAP2-R (5 mM) (SEQ ID NO. 08)

5 μl of 10× PCR buffer (TAKARA)

0.25 μl of Taq polymerase (TAKARA)

27.8 μl of distilled water

The PCR amplification with SEQ ID No. 07 and SEQ ID No. 08 resulted in a 777 Bp fragment which consists of the promoter sequence of the PAP2 gene from Lycopersicon esculentum (SEQ ID 09). This PCR product was cloned into the PCR cloning vector pCR2.1 (Invitrogen) using standard methods, and the clone pCRLEPAP2 was obtained.

This clone was therefore used for cloning into the expression vektor pJIT117 (Guerineau et al. 1988, Nucl. Acids Res. 16:11380). pJIT117 was modified by replacing the 35S terminator by the CAT terminator (cathepsin D inhibitor gene) of Solanum tuberosum (position 9-34 of database entry AX696004, SEQ 16 of patent WO03008596).

The DNA fragment which contains the CAT terminator region was produced by PCR using the genomic DNA from Solanum tuberosum (preparation according to Galbiati M et al. (2000) Funct Integr Genomics: 25-233) and the primers CAT-1 (SEQ ID No. 25) and CAT-2 (SEQ ID No. 26).

The PCR conditions were as follows:

The PCR for amplifying the DNA which contains the cathepsin D inhibitor gene (CAT) terminator region (SEQ ID 27) took place in a 50 μl reaction mixture which comprised:

2 μl of a preparation of Solanum tuberosum DNA (prepared as described above)

5 μl of 10× dNTP mix (2 mM)

5 μl of CAT-1 (SEQ ID No. 25) (5 mM)

5 μl of CAT-2 (SEQ ID No. 26) (5 mM)

5 μl of 10× PCR buffer (TAKARA)

0.25 μl of Pfu polymerase (TAKARA)

27.8 μl distilled water

The PCR was carried out with the following cycle conditions:

| | | | |
|---|---|---|---|
| 1x | 94° C. | 2 | minutes |
| 35x | 94° C. | 1 | minute |
| | 50° C. | 1 | minute |
| | 72° C. | 1 | minute |
| 1x | 72° C. | 10 | minutes |

The 237 bp amplicon (SEQ ID NO. 27) was cloned into the PCR cloning vector pCR 2.1 (Invitrogen) using standard methods, and the plasmid pCAT was obtained.

The CAT terminator was cloned into the pJIT117 vector by isolating the SalI/SpeI

fragment from pCAT and ligating into the SalI/SpeI-cleaved vector pJIT117. The clone is called pJCAT.

The LEPAP2 promoter was cloned into the vector pJCAT by isolating the 777 bp KpnI/HindIII fragment which encodes the LEPAP2 promoter from pCRLEPAP2 and ligating into the KpnI/HindIII-cleaved vector pJCAT. The resulting clone, which comprises the LEPAP2 promoter in the correct orientation with the rbcs transit peptide, is called pJLEPAP2CAT.

### Example 2

Amplification of a DNA which corresponds to the complete primary sequence of the BNPAP2 promoter from Brassica napus.

The DNA which codes for the PAP2 promoter of the plant Brassica napus was isolated from Brassica napus by thermal-interlaced (TAIL-) PCR (Liu et al. 1995 Plant J 457-463, Tsugeki et al. 1996 Plant J 479-489).

TAIL-PCR can be used to isolate unknown DNA fragments which flank a known DNA sequence. In this example, sequences which are located in the genome upstream of the corresponding 5′ end of the known oilseed rape cDNA sequence AF290564 (“Brassica rapa plastid-lipid associated protein PAP2 mRNA, complete cds; nuclear gene for plastid product”) (SEQ ID No. 10) were isolated. Three different primers with antisense orientation to the oilseed rape cDNA AF290564 were derived. These primers were employed singly in consecutive PCR reactions with any degenerate primer.

The following mastermix (data per reaction mixture) is employed for the first TAIL-PCR reaction.

11 μl of sterile H₂O (double distilled)

2 μl of primer stock solution of the specific primer BNPAP2-TAIL-1 (5 mM) (SEQ ID No. 11)

3 μl of AD1 primer stock solution (20 mM) (SEQ ID No. 14)

2 μl of 10× PCR buffer (TAKARA)

2 μl of 10× dNTP mix (2 mM)

0.2 μl of Taq polymerase (TAKARA)

20.2 μl of this mastermix are pipetted into 1 μl of a preparation of genomic DNA from Brassica napus (preparation according to Galbiati M et al. (2000) Funct Integr Genomics: 25-34) in a PCR vessel and thoroughly mixed by pipetting. The primary PCR reaction is carried out under the following conditions:
94° C. for 1 min
four cycles at 94° C. for 10 s, 62° C. for 1 min and 72° C. for 150 s
94° C. for 10 s, 25° C. for 3 min, 0.20 C/s to 72° C. and 72° C. for 150 s
fourteen cycles at 94° C. for 10 s, 69° C. for 1 min, 72° C. for 150 s, 94° C. for 10 s, 68° C. for 1 min, 72° C. for 150 s, 94° C. for 10 s, 44° C. for 1 min and 720° C. for 150 s
72° C. for 5 min, then 4° C. until used further.

The product of the primary PCR reaction is diluted 1:50, and 1 μl portions of the dilution are used for a second PCR reaction (secondary PCR). The following mastermix (data per reaction mixture) is employed for this:

11 μl of sterile H₂O (double distilled)

2 μl of primer stock solution of the specific primer IBNPAP2-TAIL-2 (5 mM) (SEQ ID No. 12)

2 μl of AD1 primer stock solution (20 mM) (SEQ ID No. 14)

2 μl of 10× PCR buffer (TAKARA)

2 μl of 10× dNTP mix (2 mM)

0.2 μl of Taq polymerase (TAKARA)

19.2 μl portions of the second mastermix are added to 1 μl portions of the primary PCR product diluted 1:50, and the secondary PCR is carried out under the following conditions:
11 cycles at 94° C. for 10 s, 64° C. for 1 min, 72° C. for 150 s, 94° C. for 10 s, 64° C. for 1 min, 72° C. for 150 s, 94° C. for 10 s, 44° C. for 1 min, 72° C. for 150 s, 72° C. for 5 min, then 4° C. until used further.

The PCR product of the secondary PCR reaction is diluted 1:10, and 1 μl portions of the dilution are used for a third PCR reaction (tertiary PCR). The following mastermix (data per reaction mixture) is employed for this:

18 μl of sterile H₂O (double distilled)

3 μl of 10× PCR buffer (TAKARA)

3 μl of 10× dNTP mix (2 mM)

2 μl of primer stock solution of the specific primer BNPAP2-TAIL-3 (5 mM) (SEQ ID No. 13)

3 μl of AD1 primer stock solution (20 mM) (SEQ ID No. 14)

0.5 μl of Taq polymerase (TAKARA)

29.5 μl portions of this mastermix are added to 1 μl portions of the secondary PCR product diluted 1:10, and the tertiary PCR is carried out under the following conditions:
19 cycles at 94° C. for 15 s, 44° C. for 1 min, 72° C. for 150 s,
72° C. for 5 min, then 4° C. until used further.

The PCR amplification with SEQ ID No. 13 and SEQ ID No. 14 resulted in a 1564 bp fragment which consists of the promoter and a short segment at the 5′ terminus of the coding sequence of the BNPAP2 gene of Brassica napus (Seq ID No. 15). The PCR product was cloned into the PCR cloning vector PCR 2.1 (Invitrogen) using standard methods, and the clone pCRBNPAP2TAIL was obtained.

The primers BNPAP2-F2 (SEQ ID NO. 16) and BNPAP2-R2 (SEQ ID NO. 17) were derived on the basis of the sequence of the insert of pCRBNPAP2TAIL in order to amplify the correct sequence of the BNPAP2 promoter of Brassica napus (without the mutations introduced owing to multiple rounds of TAIL-PCR). For this purpose, these primers were employed in a specific PCR reaction with genomic DNA from Brassica napus as template.

The PCR conditions were as follows:

2 μl of a preparation of Brassica napus DNA (prepared as described above)

5 μl of 10× dNTP mix (2 mM)

5 μl of BNPAP2-F2 (SEQ ID NO. 16) (5 mM)

5 μl of BNPAP2-R2 (SEQ ID NO. 17) (5 mM)

5 μl of 10× PCR buffer (TAKARA)

0.25 μl of Taq polymerase (TAKARA)

27.8 μl of distilled water

The PCR amplification with SEQ ID No. 16 and SEQ ID No. 17 resulted in a 960 bp fragment which corresponds to the promoter of the PAP2 gene of Brassica napus (Seq ID No. 18). The PCR product was cloned into the PCR cloning vector pCR 2.1 (Invitrogen) using standard methods, and the clone pCRBNPAP2 was obtained.

This clone was subsequently used for cloning into the expression vector pJIT117 (Guerineau et al. 1988, Nucl Acids Res. 16: 11380). The cloning took place by isolating the 958 Bp KpnI/HindIII fragment from pCRBNPAP2 and ligating into the KpnII/HindIII-cleaved vector pJIT117. The resulting clone which comprises the BNPAP2 promoter in the correct orientation with the rbcs transit peptide is called pJBNPAP2.

### Example 3

Amplification of a DNA which corresponds to the complete primary sequence of the BNPAPX promoter from Brassica napus.

The DNA which codes for the PAPX promoter from Brassica napus was amplified from Brassica napus by PCR. The primers BNPAP2-F2 (SEQ ID NO. 19) and BNPAP2-R1 (SEQ ID NO. 20) were derived on the basis of the sequence of the insert of pCRBNPAP2TAIL. For this purpose, these primers were employed in a specific PCR reaction with genomic DNA from Brassica napus as template.

The PCR conditions were as follows:

2 μl of a preparation of Brassica napus DNA (prepared as described above)

5 μl of 10× dNTP mix (2 mM)

5 μl of BNPAP2-F2 (SEQ ID NO. 19) (5 mM)

5 μl of BNPAP2-R1 (SEQ ID NO. 20) (5 mM)

5 μl of 10× PCR buffer (TAKARA)

0.25 μl of Taq polymerase (TAKARA)

27.8 μl of distilled water

The PCR amplification with SEQ ID No. 19 and SEQ ID No. 20 resulted in a 1043 Bp fragment which consists of the promoter of the PAPX gene of Brassica napus (SEQ ID No. 21). The PCR product was cloned into the PCR cloning vector pCR 2.1 (Invitrogen) using standard methods, and the clone pCRBNPAPX was obtained.

This clone was subsequently used for cloning into the expression vector pJIT117 (Guerineau et al. 1988, Nucl. Acids Res. 16: 11380). The expression vector pJIT117 was modified by replacing the 35S terminator the OCS terminator (octopine synthase) of the Ti plasmid pTi15955 of Agrobacterium tumefaciens (database entry X00493 from position 12,541-12,350, Gielen et al. (1984) EMBO J. 3 835-846).

The DNA fragment which contains the OCS terminator region (SEQ ID No. 30) was prepared by PCR using the plasmid pHELLSGATE (database entry AJ311874, Wesley et al. (2001) Plant J. 27 581-590, isolated from E. coli by standard methods), and the primers OCS-1 (SEQ ID No. 28) and OCS-2 (SEQ ID No. 29).

The PCR for amplifying the DNA which contains the octopine synthase (OCS) terminator region (SEQ ID 30) took place in a 50 μl reaction mixture which comprised:

2 μl of a preparation of pHELLSGATE plasmid DNA

5 μl of 10× dNTP mix (2 mM)

5 μl of OCS-1. (SEQ ID No. 28) (5 mM)

5 μl of OCS-2 (SEQ ID No. 29) (5 mM)

5 μl of 10× PCR buffer (TAKARA)

0.25 μl of Pfu polymerase (TAKARA)

27.8 μl of distilled water

The PCR was carried out under the following cycle conditions:

| | | | |
|---|---|---|---|
| 1x | 94° C. | 2 | minutes |
| 35x | 94° C. | 1 | minute |
| | 50° C. | 1 | minute |
| | 72° C. | 1 | minute |
| 1x | 72° C. | 10 | minutes |

The 204 bp amplicon was cloned into the PCR cloning vector pCR 2.1 (Invitrogen) using standard methods, and the plasmid pOCS was obtained.

Sequencing of the pOCS clone confirmed a sequence which agrees with a sequence segment from the Ti plasmid pTi15955 of Agrobacterium tumefaciens (database entry X00493) from position 12,541 to 12,350.

The OCS terminator was cloned into the pJIT117 vector by isolating the SalI/SpeI fragment from pOCS and ligating into the SalI-SpeI-cleaved vector pJIT117. The clone is called pJOCS.

The BNPAPX promoter was cloned into the pJOCS vector by isolating the KpnI/HindIII fragment which codes for the BNPAPX promoter from pCRBNPAPX and ligating into the KpnI/HindIII-cleaved vector pJOCS. The resulting clone, which comprises the BNPAPX promoter in the correct orientation with the rbcs transit peptide, is called pJBNPAPXOCS.

### Example 4

Amplification of a cDNA which codes for the complete primary sequence of the ketolase from Haematococcus pluvialis Flotow em. Wille.

The cDNA which codes for the ketolase from Haematococcus pluvialis was obtained from Haematococcus pluvialis suspension cultures (strain 192.80 of the “Sammlung von Algenkulturen der Universität Göttingen”) by the method described below.

Total RNA was prepared from Haematococcus pluvialis by isolating cells from a suspension culture which was cultured at room temperature with indirect daylight for 2 weeks. The medium used was “Haematococcus medium” (1.2 g/l sodium acetate, 2 g/l yeast extract, 0.2 g/l MgCl₂×6H₂O, 0.02 g/l CaCl₂×2H₂O; pH 6.8; after autoclaving; addition of 400 mg/l L-asparagine, 10 mg/l FeSO₄×H₂O). The Haematococcus pluvialis cells were harvested from the abovementioned culture, frozen in liquid nitrogen and powdered in a mortar. Then 100 mg of the frozen, powdered algal cells were transferred into a reaction vessel and resuspended in 0.8 ml of Trizol buffer (LifeTechnologies). This suspension was extracted with 0.2 ml of chloroform. After centrifugation at 12000×g for 15 minutes, the aqueous supernatant was removed and transferred into a new reaction vessel. The RNA was precipitated with one part by volume of isopropanol, the pellet after centrifugation and removal of the supernatant was washed with 75% ethanol and then dissolved in DEPC water (DEPC water; incubation of water with 1/1000 part by volume of diethyl pyrocarbonate at room temperature overnight and then autoclave). The RNA concentration was determined by photometry.

For the cDNA synthesis, 2.5 μg of total RNA were denaturated at 60° C. for 10 min, cooled on ice for 2 min and transcribed into cDNA using an antisense-specific primer (PR1 SEQ ID NO. 22) by means of a cDNA kit (“Ready-to-go-you-prime-beads”, Pharmacia Biotech) in accordance with the manufacturer's instructions.

The nucleic acid which codes for a ketolase from Haematococcus pluvialis (strain 192.80) was amplified by polymerase chain reaction (PCR) from Haematococcus pluvialis using a sense-specific primer (PR1 SEQ ID NO. 22) and an antisense-specific primer (PR2 SEQ ID NO. 23) with the cDNA from the synthesis reaction described above as template.

The PCR conditions were as follows:

4 μl of Haematococcus pluvialis cDNA (prepared as described above)

5 μl of 10× dNTP Mix (2 mM)

5 μl of PR1 (SEQ ID No.22) (5 mM)

5 μl of PR2 (SEQ ID No. 23) (5 mM)

5 μl of 10× PCR buffer (TAKARA)

0.25 μl of Pfu polymerase (TAKARA)

25.8 μl of distilled water

The PCR was carried out under the following cycle conditions:

| | | | |
|---|---|---|---|
| 1x | 94° C. | 2 | minutes |
| 35x | 94° C. | 1 | minute |
| | 53° C. | 2 | minutes |
| | 72° C. | 3 | minutes |
| 1x | 72° C. | 10 | minutes |

The PCR amplification with SEQ ID NO. 22 and SEQ ID NO. 23 resulted in a 997 bp fragment which codes for a protein consisting of the complete primary sequence of a ketolase (SEQ ID NO. 24). The PCR product was cloned into the PCR cloning vector pCR2.1 (Invitrogen) using standard methods, and the clone pCRKETO2 was obtained.

Sequencing of the clone pCRKETO2 with the T7 and SP6 primers confirmed a sequence which differs from the published sequence X86782 by only one base in each of the three codons 73, 114 and 119. These nucleotide exchanges were reproduced in an independent amplification experiment and thus represent the nucleotide sequence of the ketolase of the Haematococcus pluvialis strain 192.80 used.

### Example 5

Production of expression vectors for flower-specific expression of the ketolase from Haematococcus pluvialis in Tagetes erecta.

The clone pCRKETO2 was used for the cloning into the expression vector pJLEPAP2CAT (see above). The cloning took place by isolating the 997 Bp SphI fragment from pCRKETO2 and ligating into the SphI-cleaved vector pJLEPAP2. The clone which comprises the Haematococcus pluvialis ketolase in the correct orientation as N-terminal translational fusion with the rbcs transit peptide is called pLEPAPBKT.

An expression vector for Agrobacterium-mediated transformation of the LE PAP2-controlled ketolase from Haematococcus pluvialis into Tagetes erecta was prepared using the binary vector pSUN5 (WO02/00900).

The expression vector pS5LEPAP2BKT was prepared by ligating the KpnI fragment from pLEPAPBKT with the KpnI-cleaved vector pSUN5. pS5LEPAP2BKT contains the LEPAP2-promoter from Lycopersicon esculentum, the rbcS transit peptide from Pisum sativum, the ketolase from Haematococcus pluvialis and the polyadenylation signal of the octopine synthase from Agrobaceriurn tumefaciens.

The clone pCRKETO2 was further used for cloning into the expression vector pJBNPAP2 (see above). The cloning took place by isolating the 997 Bp SphI fragment from pCRKETO2 and ligating into the SphI-cleaved vector pJBNPAP2. The clone which comprises the Haematococcus pluvialis ketolase in the correct orientation as N-terminal translational fusion with the rbcs transit peptide is called pBNPAP2BKT.

An expression vector for Agrobacterium-mediated transformation of the BN PAP2-controlled expression of the ketolase from Haematococcus pluvialis in Tagetes erecta was prepared using the binary vector pSUN5 (WO02100900).

The expression vector pS5BNPAP2BKT was prepared by ligating the KpnI fragment from pBNPAP2BKT with the KpnI-cleaved vector pSUN5. pS5BNPAP2BKT 1 contains the BNPAP2 promoter from Brassica napus, the rbcS transit peptide from Pisum sativum, the ketolase from Haematococcus pluvialis and the polyadenylation signal of the octopine synthase from Agrobacterium tumefaciens.

The clone pCRKET02 was therefore used for cloning into the expression vector pJBNPAPXOCS (see above). The cloning took place by isolating the 997 Bp SphI fragment from pCRKETO2 and ligating into the SphI-cleaved vector pJBNPAPX. The clone which comprises the sequence of the Haematococcus pluvialis ketolase in the correct orientation as N-terminal translational fusion with the rbcs transit peptide is called pBNPAPXBKT.

Preparation of an expression vector for the Agrobacterium-mediated transformation of the ketolase from Haematococcus pluvialis under the expression control of the BN PAPX promoter in Tagetes erecta took place using the binary vector pSUN5 (WO02/00900).

The expression vector pS5BNPAPXBKT was prepared by ligating the KpnI fragment from pBNPAPXBKT into the KpnI-cleaved vector pSUN5. pS5BNPAPXBKT contains the BNPAPX promoter from Brassica napus, the rbcS transit peptide from Pisum sativum, the ketolase from Haematococcus pluvialis and the polyadenylation signal of the octopine synthase from Agrobacterium tumefaciens.

### Example 6

Production of Transgenic Tagetes Plants

The transformation of Tagetes essentially follows the method described in WO 01/46445. Tagetes seeds of the line “Zitronenprinz” (obtained from the IPK-Genbank, Gatersieben, Germany; listed there under TAG76; also obtainable from Zierpflanzensammlung; Erfurt: collection number: 8184,00) are sterilized and placed on germination medium (MS medium; Murashige and Skoog, Physiol. Plant. 15(1962), 473-497) pH 5.8, 2% sucrose). The germination takes place in a temperature/light/time interval of 18 to 28° C./20-200 μE/m²s for 3 to 16 weeks, but preferably at 21° C., 20 to 70 μE/m²s, for 4 to 8 weeks.

All the leaves of the plants which have developed by then in vitro are harvested and cut transverse to the mid rib. The leaf explants produced thereby with a size of 10 to 60 mm² are stored during the preparation in liquid MS medium at room temperature for a maximum of 2 h.

Any Agrobacterium tumefaciens strain, but preferably a supervirulent strain such as, for example, EHA105 with an appropriate binary plasmid which may harbor a selection marker gene (preferably bar or pat), and one or more trait or reporter genes is (for example pS5BNPAPXBKT and pS5LEPAP2BKT) cultured overnight and used for coculturing with the leaf material. The culturing of the bacterial strain can take place as follows:

A single colony of the appropriate strain is inoculated in YEB (0.1% yeast extract, 0.5% beef extract, 0.5% peptone, 0.5% sucrose, 0.5% magnesium sulfate×7 H₂O ) with 25 mg/l kanamycin and cultured at 28° C. for 16 to 20 h. The bacterial suspension is then harvested by centrifugation at 6000 g for 10 min and resuspended in liquid MS medium in such a way that an OD₆₀₀ of about 0.1 to 0.8 resulted. This suspension is used for the coculturing with the leaf material.

Immediately before the coculturing, the MS medium in which the leaves have been stored is replaced by the bacterial suspension. Incubation of the leaflets in the suspension of Agrobacteria took place at room temperature with gentle shaking for 30 min. The infected explants are then placed on an MS medium which has been solidified with agar, e.g. 0.8% plant agar (Duchefa, NL) with growth regulators such as, for example, 3 mg/l benzylaminopurine (BAP) and 1 mg/l indolylacetic acid (IAA). The orientation of the leaves on the medium has no significance. The explants are cultivated for 1 to 8 days, but preferably for 6 days, and in this case the following conditions can be used: light intensity: 30 to 80 mmol/m²×sec, temperature. 22 to 24° C., 16/8 hours light/dark alternation. The cocultivated explants are then transferred to fresh MS medium, preferably with the same growth regulators, this second medium additionally comprising an antibiotic to suppress bacterial growth. Timentin in a concentration of 200 to 500 mg/l is very suitable for this purpose. The second selective component employed is one to select for successful transformation. Phosphinothricin in a concentration of 1 to 5 mg/l selects very efficiently, but other selective components are conceivable according to the process used.

After from one to three weeks in each case, the explants are transferred to fresh medium until shoot buds and small shoots develop, which are then transferred to the same basal medium including Timentin and PPT or alternative components with growth regulators, namely, for example, 0.5 mg/l indolylbutyric acid (IBA) and 0.5 mg/l gibberillic acid GA₃, for rooting. Rooted shoots can be transferred to a glass house.

In addition to the methods described, the following advantageous modifications are possible:
Before the explants are infected with the bacteria, they can be preincubated for 1 to 12 days, preferably 3 to 4, on the medium described above for the coculture. This is followed by the infection, coculture and selective regeneration as described above.
The pH for the regeneration (normally 5.8) can be reduced to 5.2. This improves control of the growth of Agrobacteria.
Addition of AgNO₃ (3-10 mg/l) to the regeneration medium improves the condition of the culture, including the regeneration itself.
Components which reduce phenol formation and are known to the skilled worker, such as, for example, citric acid, ascorbic acid, PVP and many others, have a positive effect on the culture.
Liquid culture medium can also be used for the overall process. The culture can also be incubated on commercially available supports which are positioned on the liquid medium.

The following lines were obtained by the transformation method described above using the following expression constructs:

After transformation with the binary vector pSSLEPAP2BKT, for example, the following plants were obtained: MS301-10, MS301-16, MS301-19.

After transformation with the binary vector pS5BNPAPXBKT, for example, the plants referred to hereinafter as MS259-11, MS259-23, M259-28 were obtained.

### Example 7

Enzymatic lipase-catalyzed hydrolysis of carotenoid esters from plant material and identification of the carotenoids

General Procedure

a) Ground plant material, e.g. petal material (fresh weight 30-100 mg), is extracted with 100% acetone (three times 500 μl; shake for about 15 minutes each time). The solvent is evaporated. Carotenoids are then taken up in 495 μl of acetone, 4.95 ml of potassium phosphate buffer (100 mM, pH7.4) are added and thoroughly mixed. This is followed by addition of about 17 mg of bile salts (Sigma) and 149 μl of an NaCl/CaCl₂ solution (3M NaCl and 75 mM CaCl₂). The suspension is incubated at 37° C. for 30 minutes. For the enzymatic hydrolysis of the carotenoid esters, 595 μl of a lipase solution (50 mg/ml lipase type 7 from Candida rugosa (Sigma)) are added and incubated at 37° C. with shaking. After about 21 hours, 595 μl of lipase are again added, and incubation is continued at 37° C. for at least 5 hours. Approximately about 700 mg of Na₂SO₄ are then dissolved in the solution. After addition of 1800 μl of petroleum ether, the carotenoids are extracted into the organic phase by vigorous mixing. This extraction is repeated until the organic phase remains colorless. The petroleum ether fractions are combined, and the petroleum ether is evaporated. Free carotenoids are taken up in 100-120 μl of acetone. Free carotenoids can be identified by means of HPLC and C30 reverse phase column on the basis of the retention time and UV-VIS spectra.

The bile salts or bile acid salts used are 1:1 mixtures of cholate and deoxycholate.

b) Procedure for Workup if Only Small Amounts of Carotenoid Esters are Present in the Plant Material

Alternatively, the carotenoid esters can be hydrolyzed by Candida rugosa lipase after separation by means of thin-layer chromatography. For this purpose, 50-100 mg of plant material are extracted three times with about 750 μl of acetone. The solvent extract is concentrated in a rotary evaporator in vacuo (raised temperatures of 40-50° C. are tolerable). This is followed by addition of 300 μl of petroleum ether: acetone (ratio 5:1) and thorough mixing. Suspended materials are sedimented by centrifugation (1-2 minutes). The upper phase is transferred into a new reaction vessel. The remaining residue is again extracted with 200 μl of petroleum ether:acetone (ratio 5:1), and suspended materials are removed by centrifugation. The two extracts are put together (volume 500 μl) and the solvents are evaporated. The residue is resuspended in 30 μl of petroleum ether:acetone (ratio 5:1) and loaded onto a thin-layer plate (silica gel 60, Merck). If more than one loading is necessary for preparative-analytical purposes, several aliquots each with a fresh weight of 50-100 mg should be prepared in the described manner for the separation by thin-layer chromatography.

The thin-layer plate is developed in petroleum ether:acetone (ratio 5:1). Carotenoid bands can be identified visually on the basis of their color. Individual carotenoid bands are scraped off and can be pooled for preparative-analytical purposes. The carotenoids are eluted from the silica material with acetone; the solvent is evaporated in vacuo. To hydrolyze the carotenoid esters, the residue is dissolved in 495 μl of acetone, and 17 mg of bile salts (Sigma), 4.95 ml of 0.1M potassium phosphate buffer (pH 7.4) and 149 μl (3M NaCl, 75 mM CaCl₂) are added. After thorough mixing, the mixture is equilibrated at 37° C. for 30 min. This is followed by addition of 595 μl of Candida rugosa lipase (Sigma, stock solution of 50 mg/ml in 5 mM CaCl₂). Incubation with lipase takes place overnight with shaking at 37° C. After about 21 hours, the same amount of lipase is again added; incubation is continued with shaking at 37° C. for at least 5 hours. This is followed by addition of 700 mg of Na₂SO₄ (anhydrous); extraction is carried out with 1800 μl of petroleum ether for about 1 minute, and the mixture is centrifuged at 3500 revolutions/minute for 5 minutes. The upper phase is transferred into a new reaction vessel, and the extraction is continued until the upper phase is colorless. The combined petroleum ether phase is concentrated in vacuo (temperatures of 40-50° C. are possible). The residue is dissolved in 120 μl of acetone, possibly using ultrasound. The dissolved carotenoids can be separated by HPLC using a C30 column and quantified by means of reference substances.

### Example 8

HPLC Analysis of Free Carotenoids

The samples obtained by the procedures in Example 7 are analyzed under the following conditions:

HPLC Conditions:

Separating column: Prontosil C30 column, 250×4.6 mm, (Bischoff, Leonberg, Germany)

Flow rate: 1.0 ml/min

Eluents: mobile phase A—100% methanol
mobile phase B—80% methanol, 0.2% ammonium acetate
mobile phase C—100% t-butyl methyl ether

Detection: 300-530 nm

Gradient Profile:

| | | % mobile | % mobile | % mobile |
|---|---|---|---|---|
| Time | Flow rate | phase A | phase B | phase C |
| 1.00 | 1.0 | 95.0 | 5.0 | 0 |
| 12.00 | 1.0 | 95.0 | 5.0 | 0 |
| 12.10 | 1.0 | 80.0 | 5.0 | 15.0 |
| 22.00 | 1.0 | 76.0 | 5.0 | 19.0 |
| 22.10 | 1.0 | 66.5 | 5.0 | 28.5 |
| 38.00 | 1.0 | 15.0 | 5.0 | 80.0 |
| 45.00 | 1.0 | 95.0 | 5.0 | 0 |
| 46.0 | 1.0 | 95.0 | 5.0 | 0 |

Some typical retention times for carotenoids produced according to the invention are, for example:

violaxanthin about 11.7 min, astaxanthin about 17.7 min, adonixanthin about 19 min, adonirubin about 19.9 min, zeaxanthin about 21 min;

The plants obtained for example after transformation with the binary vector pS5BNPAPXBKT are referred to hereinafter as MS259; the plants obtained after transformation with the binary vector pS5LEPAP2BKT are referred to as MS301. These plants accumulate newly formed ketocarotenoids which were not previously present in the petals of Tagetes.

TABLE 1
Carotenoids in the petals of transgenic Tagetes which are designated MS259 and MS301

| Plant | Epoxides | Asta | Lutein | Phoenico | Zea | Cantha | 3′-Hydroxy | b-Crypto | b-Carotin | Ketos | Asta purity |
|---|---|---|---|---|---|---|---|---|---|---|---|
| MS301-10 | 6.8 | 0.31 | 88.3 | 0.06 | 1.0 | | 0.02 | 1.2 | 2.8 | 0.4 | 79 |
| MS301-16 | 6.4 | 0.15 | 90.2 | 0.04 | 0.8 | | 0.02 | 0.4 | 2.2 | 0.2 | 73 |
| MS301-19 | 6.4 | 0.08 | 89.5 | 0.02 | 0.9 | | 0.02 | 0.6 | 2.5 | 0.1 | 67 |
| MS259-11 | 7.1 | 1.6 | 57.6 | 0.2 | 0.8 | 0.02 | 0.03 | 0.2 | 15.4 | 1.9 | 87 |
| MS259-23 | 6.6 | 2.0 | 46.5 | 0.1 | 0.6 | 0.03 | 0.01 | 0.2 | 27.0 | 2.2 | 93 |
| MS259-28 | 6.5 | 2.5 | 48.6 | 0.2 | 0.8 | 0.02 | 0.01 | 0.3 | 20.1 | 2.7 | 91 |

The data represent the percentage proportions of individual carotenoids in a carotenoid extract which was produced with fresh petals from flowers in full bloom. All the data are thus based on the fresh weight.
Key:
“Epoxides” means the total amount of the epoxides violaxanthin, antheraxanthin and neoxanthin
“Asta” refers to astaxanthin
“Phoenico” refers to phoenicoxanthin, also synonymous with adonirubin
“Zea” refers to zeaxanthin
“Cantha” refers to canthaxanthin
“3′-Hydroxy” refers to 3′-hydroxyechinenone
“b-Crypto” refers to beta-cryptoxanthin
“Ketos” means the total amount of all the ketocarotenoids (canthaxanthin, phoenicoxanthin, astaxanthin, adonixanthin, echinenone and 3′- and 3-hydroxyechinenone.

1. A plastid-lipid associated protein promoter (PAP promoter) for heterologous expression of a gene in a plant of the genus Tagetes.

2. The PAP promoter of claim 1, wherein the expression takes place specifically in flowers, especially in petals.

3. The PAP promoter of claim 1, comprising
A1) the nucleic acid sequence of SEQ ID NO: 9, 18 or 21,
A2) a sequence derived from the nucleic acid sequence of SEQ ID NO: 9, 18 or 21 by substitution, insertion or deletion of nucleotides and having an identity of at least 60% at the nucleic acid level with the respective sequence of SEQ ID NO: 9, 18 or 21,
A3) a nucleic acid sequence which hybridizes with the nucleic acid sequence of SEQ ID NO: 9, 18 or 21 under stringent conditions, or
A4) functionally equivalent fragments of the sequences under A1), A2) or A3).

4. The PAP promoter of claim 1, wherein the PAP promoter is functionally linked to a ketolase gene.

5. A genetically modified plant of the genus Tagetes, wherein the expression rate of at least one gene is increased as compared with the wild type and wherein the increased expression rate is caused by regulating the expression of said gene in a plant by the PAP promoter claim 1.

6. The genetically modified plant of claim 5, wherein the regulation of the expression of the gene is achieved by
a) introducing one or more PAP promoters into the genome of the plant, so that expression of one or more endogenous genes takes place under the control of the introduced PAP promoters,
b) introducing one or more genes into the genome of the plant, so that expression of one or more of the introduced genes takes place under the control of the endogenous PAP promoters, or
c) introducing one or more nucleic acid constructs comprising at least one PAP promoter and, functionally linked to, one or more genes to be expressed into the plant.

7. A process for producing biosynthetic products by cultivating genetically modified plants of the genus Tagetes of claim 5.

8. A process for producing carotenoids by cultivating genetically modified plants of claim 5, wherein the genes to be expressed comprise at least one ketolase.

9. The process of claim 8, wherein a ketolase from Haematococcus is used.

10. The process of claim 8 for producing astaxanthin and astaxanthin derivatives.

11. The process of claim 10, wherein the genetically modified plants or parts of plants are harvested after the cultivation, and then the carotenoids are isolated from the genetically modified plants or parts of plants.

12. A method for heterologous expression of a gene in a plant of the genus Tagetes, comprising functionally linking a gene to a plastid-lipid associated protein promoter (PAP promoter) and expressing said gene in a plant.

13. The method of claim 12, wherein the expression takes place specifically in flowers, especially in petals.

14. The method of claim 12, wherein the PAP promoter
A1) comprises the nucleic acid sequence of SEQ ID NO: 9, 18 or 21, or
A2) comprises a sequence derived from these sequences by substitution, insertion or deletion of nucleotides and having an identity of at least 60% at the nucleic acid level with the respective sequence of SEQ ID NO: 9, 18 or 21, or
A3) a nucleic acid sequence which hybridizes with the nucleic acid sequence of SEQ ID NO: 9, 18 or 21 under stringent conditions, or
A4) functionally equivalent fragments of the sequences under A1), A2) or A3).

15. The method of claim 12, wherein the PAP promoter is functionally linked to a ketolase gene.


SEQUENCE LISTING (30 sequences)

SEQ ID NO: 1 - DNA, 812 bp, Lycopersicon esculentum
  promoter (1)..(812)
ttttcttcatttcatatttacaaaaaatagcattctgctctttctctgtttcactctgaaaatggcttccatctcttctctcaatcaaattccttgcagaactctccaaattacatcccaatattcaaaacccacctccaaaatctcaactttacccatctcctccacgaatttcccatcaaaaacagaactacacagagcaatttcagtcaaagaattcacatacccaaaaccaaaattcacagctcaagccacaaattacgacaaggaagatgagtgggggccggaagtggagaaaataagtccgggtggagtagcggttgtggatgaagaaccaccaaaggagccaagtgaaattgagttgctgaagaagcaattggcggattctttttatggaaccaataggggtttgagtgctagcagtgagactagggcggaaatcgttgaactcatcacacagcttgagtcgaagaaccctaatccagcacctactgaagccttgactcttctcaacggcaaatggattcttgcgtacacatctttttcgggtttgttccccttgttgtcacggggcaatctgcttctggttcgagttgaagagatttcacagactatcgattctgagagtttcactgtccaaaactctgttgtctttgctggacctttagctacaacttccattagtaccaatgccaaattcgaagtcagaagtccaaaacgcgtccagattaaattcgaagaaggcataattggaaacacccagttgacagattccatcgtattgcctgagaaacgtgaattcttgggacaaa

SEQ ID NO: 2 - DNA, 22 bp, Artificial sequence
  Description of Artificial sequence PCR primer
gctcctttggtggttcttcatc

SEQ ID NO: 3 - DNA, 22 bp, Artificial sequence
  Description of Artificial sequence PCR primer
cgctactccacccggacttatt

SEQ ID NO: 4 - DNA, 22 bp, Artificial sequence
  Description of Artificial sequence PCR primer
cccactcatcttccttgtcgta

SEQ ID NO: 5 - DNA, 15 bp, Artificial sequence
  Description of Artificial sequence PCR primer
ntcgastwtsgwgtt

SEQ ID NO: 6 - DNA, 997 bp, Lycopersicon esculentum
  promoter (1)..(997)
gtcgagtttggagttataaatgaattaaagttagtagttaaagaattctattattagccaatgtagtttgattttttgatatcttaatttataatttttgcggtgtcgtgaagtttcatgactttagttatagacatcaatcaacgactattcaattacatattttagttatacatatcaatcaataattaatcatatgtatgaatttagttataaagttcaatcaataattatttaactacatcatcccaatgttatcttctttttatcatatattttaggatattttttttcgtgagaaaaaaaccatcattaataattctcaaatatactttttaccatatatttagaattatttaattttttaagataatcaatgttataagagattacataaaaatcaagaaacatgaagtaataggtattgataggttttcttttcgtttattcacactctcaactagtaaatattttttatccaacattcaatattcatttaacatctgatatatttaaatttacgtcacatagaactattcagaaatatttagtgaaggaataaataactctccatactgatagtctcaaattaataaatatactaaactaaatgaaaagggtatgtatgtctttttaccccagctatccaaggcaacacgctcgttataaaatgcatcatacagtattgtaattctcattcaatcgagcttcccctgttttcttcatttcatatttacaaaaaatagcattctgctctttctctgtttcactctgaaaatggcttccatctcttctctcaatcaaattccctgcagaactctccaaattacatcccaatattcaaaacccacctccaaaatctcaactttacccatctcctccacgaatttcccatcaaaaacagaactacacagagcaatttcagtcaaagaattcacatacccaaaaccaaaattcacagctcaagccacaaattacgacaaggaagatgagtggg

SEQ ID NO: 7 - DNA, 30 bp, Artificial sequence
  Description of Artificial sequence PCR primer
ggtaccgagttataaacgaattaaagttag

SEQ ID NO: 8 - DNA, 28 bp, Artificial sequence
  Description of Artificial sequence PCR primer
aagctttcagagtgaaacagagaaagag

SEQ ID NO: 9 - DNA, 777 bp, Artificial sequence
  Description of Artificial sequence PCR primer
ggtaccgagttataaacgaattaaagttagtagttaaagaattctattattagccaatgtagtttgattttttgatatcttaatttataatttttgcggtgtcgtgaagtttcatgactttagttatagacatcaatcaacgactattcaattacatattttagttatacatatcaatcaataattaatcatatgtatgaatttagttataaagttcaatcaataattatttaactacatcatcccaatgttatcttctttttatcatatattttaggatattttttttcgtgagaaaaaaaccatcattaataattctcaaatatactttttaccatatatttagaattatttaattttttaagataatcaatgttataagagattacataaaaatcaagaaacatgaagtaataggtattgataggttttcttttcgtttattcacactctcaactagtaaatattttttatccaacattcaatattcatttaacatctgatatatttaaatttacgtcacatagaactattcagaaatatttagtgaaggaataaataactctccatactgatagtctcaaattaataaatatactaaactaaatgaaaagggtatgtatgtctttttaccccagctatccaaggcaacacgctcgttataaaatgcatcatacagtattgtaattctcattcaatcgagcttcccctgttttcttcatttcatatttacaaaaaatagcattctgctctttctctgtttcactctgaaagctt

SEQ ID NO: 10 - DNA, 1151 bp, Brassica napus
gaataattaaaaaaacaaaacaatggcgacggttcattccttcaaccaattcccctgcaaaacccgagttcaatgcccatcaaactcaaaacccctctccaagccaccgtcttcgctggtgccgatgagcgcgctcactcgccgtccgtcgttttcccccggagaattcgccgtttctcgttctgatttcagagtcagagtcatcgatgcggaagacgagttggatccggagacgagtgagggaggaggatcggctctattgatggcggaggaagcaatcgaatccgtggaggaaacggaggttctcaagagatcgctggtggactcgttgtacggcacagaccgaggtttgagcgcatcgagcgagacgagagccgagatcggagatctaatcacacagctcgagtccaagaaccctacaccagctcccaccgatgctctcttccttctcaacggcaaatggatcctcgcgtacacatcgttcgttggtttattcccgttgctttcacgagggatcgtgccgttagttaaagtcgatgagatctcacagactatcgattcagacaacttcacggtcgagaactctgttctgttcgctggtccgttagctacaacctcgataagcaccaacgccaaattcgaaatccgtagccccaaacgcgtccaaattaagttcgaggaaggtgtgatcgggactcctcagctgacggattcgattgagatcccggagtatgttgagtttcttggtcagaagattgatcttactccgatcagagggttgcttacatctgtacaagacacggctacgtctgtggctagaaccatatcgagccagccaccgttgaaattctctttgccaggggacagtgctcagtcgtggctgctcacgacttatctggacaaggacattaggatctctagaggagatggtggaagcgtctttgtgcttatcaaagaaggaagccctctcttgaacccttgaactctttaatgccaagtacaaactaagtgtttaatattgttgttgtagtgttaatcgtttgttttaactcggatggtatggagttatgaaataatgttgtagctgctataaaaaaaatacaaaaaaagaaatatacaaatgttaagaatgtcaaaaaaaaaaaaaaaaa

SEQ ID NO: 11 - DNA, 25 bp, Artificial sequence
  Description of Artificial sequence PCR primer
tcgatgactctgactctgaaatcag

SEQ ID NO: 12 - DNA, 20 bp, Artificial sequence
  Description of Artificial sequence PCR primer
cgaagacggtggcttggaga

SEQ ID NO: 13 - DNA, 19 bp, Artificial sequence
  Description of Artificial sequence PCR primer
gggcattgaactcgggttt

SEQ ID NO: 14 - DNA, 15 bp, Artificial sequence
  Description of Artificial sequence primer
ntcgastwtsgwgtt

SEQ ID NO: 15 - DNA, 1564 bp, Brassica napus
  misc_feature (156)..(156): n is a, c, g or t
cgtcgagagagaagaaagcggactcgttagagagcggcttgagattcaaatgcattgcatgctgtgggcccaccatcaacaatacaacaactagttgctactctccacagtccaacctctagagaagacgaaatcgaagaacaaagtgaaatccanttcgttcccggatcaaacactggatcgggttcgggtatgaatctcgcgacggctttagaggcggagagatacaaccggggagaacaggcggttgatatgacgccgaggagagtgtcgttgatgagattgctggatgagacggcggagggagtgaacgatgacgggagagaggcggagatatcaacggcgtcgatggagacgttaacgggtaacgattcggtgtgctgcgtgtgtatgggaagaaagaaaggcgctgcgtttatcccctgtggacatactttttgcagagtttgctctagagagctgtggctgaaccgaggctcgtgtcctctttgcaaccgtccgatcattgagatcttagacatattttgaaaccaatcgaatggctgtaaattctcgatctaacacagtttatacattcgtttgttatgggaagatgatgtaaatttcaaaaaaatacaagatattctgttcagaaggcccattaagtcaattttaagcccaataaaccaatagtttggacgtttcctctctatcgcgcttttccgaaacgccagaaaaacgctgtcaagatatttatcgcatggtagcagatctcgaccgttaaaggaccaaagtagttggcaaactgagcgataatcagaaaacagccagacaatagtacaccgccgaagtttctagcatcgtgtcatttgcatggcgcctaattctcgtcgagaactagagataataaagagcaactttcctaaacaataatttctgtctatccctaaatatttttttttgttttttgcagagaaactttctttttcatttattagtactttccatgtgcacagagaaacaatgtattttgtttacttcaaaatttcacaaagtacaaaagcatttaaaggacattatgtgacacacgtcatacaatttaatgagctctatgactttcagtttgaagatctaattatagcattgaatcacatcgtaattacagattatgattatctgtgtgtaaaattgtactcctagtttcagaatcccttactaatcaatcctttctacattttgaaaaattagtcagcatgtgtcaaattggatttcttgccataaacaagagccaaaaaaaacccaaaacaatgatcgcctcacaccacgatgcttaagatgtctgtctgattatcgtgcctytgatcttaccgatttatgtggctaacatttattacccctcgtgatttggtttgagattacgccatttgccaaataagcagtaatggtggctataaataaggactcagtatatacatttttcttgttcactagcttcgaagtttctttcgaatagttaaaaaaaacaaacaatggcgacggttcagttcttcaaccaattcccctgccaaacccgagttcaatgccc

SEQ ID NO: 16 - DNA, 20 bp, Artificial sequence
  Description of Artificial sequence PCR primer
ggtaccaggctcgtgtcctc

SEQ ID NO: 17 - DNA, 21 bp, Artificial sequence
  Description of Artificial sequence PCR primer
aagcttttactgcttatttgg

SEQ ID NO: 18 - DNA, 960 bp, Artificial sequence
  Description of Artificial sequence PCR primer
ggtaccaggctcgtgtcctctttgcaaccgtccgatcattgagatcttagacatattttgaaaccaatcgaatggctgtaaattctcgatctaacacagtttatacattcgtttgttatgggaagatgatgtaaatttcaaaaaaatacaagatattctgttcagaaggcccattaagtcaattttaagcccaataaaccaatagtttggacgtttcctctctatcgcgcttttccgaaacgccagaaaaacgctgtcaagatatttatcgcatggtagcagatctcgaccgttaaaggaccaaagtagttggcaaactgagcgataatcagaaaacagccagacaatagtacaccgccgaagtttctagcatcgtgtcatttgcatggcgcctaattctcgtcgagaactagagataataaagagcaactttcctaaacaataatttctgtctatccctaaatatttttttttgttctttgcagagaaactttctttttcatttattagtactttccatgtgcacagagaaacaatgtattttgtttacttcaaaatttcacaaagtacaaaagcatttaaaggacattatgtgacacacgtcatacaatttaatgagctctatgactttcagtttgaagatctaattatagcattgaatcacatcgtaattacagattatgattatctgtgtgtaaaattgtactcctagtttcagaatcccttactaatcaatcctttctacattttgaaaaattagtcagcatgtgtcaaattggatttcttgccataaacaagagccaaaaaaaacccaaaacaatgatcgcctcacaccacgatgcttaagatgtctgtctgattatcgtgcctttgatcttaccaatttatgtggctaacatttattacccctcgtgatttggtttgagattacgccatttgccaaataagcagtaaaagctt

SEQ ID NO: 19 - DNA, 20 bp, Artificial sequence
  Description of Artificial sequence PCR primer
ggtaccaggctcgtgtcctc

SEQ ID NO: 20 - DNA, 24 bp, Artificial sequence
  Description of Artificial sequence PCR primer
aagctttcgaagctagtgaacaag

SEQ ID NO: 21 - DNA, 1043 bp, Brassica napus
  promoter (1)..(1043): Description of artificial sequence promoter
ggtaccaggctcgtgtcctctttgcaaccgtccgatcattgagactcttgacatattttgaaaccaatcgaatggctgtaaattctccatctaacacaaagtttttttacagtttgttgcatttcgtttgttatggaaaagtgatgtaaatttcaaaagaaaaaaaatacaagagattctgttaacaaggcccatttttagtcaattataagcccaataaacaaatagtttggacgtttcctctcaatcgcgctttccaaaacgctgtcaagatatttatcgcatggtagcagatctcgaccgttaaaggaccaaagtagttggcaaactgagcgataatcagaaaacggccagacaatagtacactgccgaagtttctagcatcggtgtcatttgcatggcgcctaattctcgtggagaagtagtagagataataaagagcaactttcctaaacaatgatttctgtctatccctaaatatatttttttttgttttttgcagagaaactttctttttcgtttattagtactttccatgtgcatacagaaacaatgtattttgtttatttcaaaatttcgcaaagtacaaaagcatttaaagggcattatgtaccattaatatataacaaaagacaacaatttaatgagctctatgatttcagtttgaagatataatcatggcattgaatcacatcgtgattataggttacgattatgtgatttttctgtgtgtaaaattgcactcctagtttcagaatccctgactaatcctttatgctttttgaaaattagccagcatgtgtcaaattggattttcttgccataaacaagagccaaaaagcccaaaacaattatcgcctcacaccacgatgcttaagatgtctgaatatcgtcctttgatcttaccaatttatgtggctaacatttattacccctcgtgatttggtttgagattacgccatttgccaaatcagcagtaatgatggctataaattagtagtcagtgtatgtacatttttcttgttcactagcttcgaaagctt

SEQ ID NO: 22 - DNA, 26 bp, Artificial sequence
  Description of Artificial sequence PCR primer
gcatgcagctagcagcgacagtaatg

SEQ ID NO: 23 - DNA, 27 bp, Artificial sequence
  Description of Artificial sequence PCR primer
gcatgctaggcaggaaccagacctcgg

SEQ ID NO: 24 - DNA, 997 bp, Haematococcus pluvialis
gcatgcagctagcagcgacagtaatgttggagcagcttaccggaagcgctgaggcactcaaggagaaggagaaggaggttgcaggcagctctgacgtgttgcgtacatgggcgacccagtactcgcttccgtcagaggagtcagacgcggcccgcccgggactgaagaatgcctacaagccaccaccttccgacacaaagggcatcacaatggcgctagctgtcatcggctcctgggccgcagtgttcctccacgccatttttcaaatcaagcttccgacctccttggaccagctgcactggctgcccgtgtcagatgccacagctcagctggttagcggcagcagcagcctgctgcacatcgtcgtagtattctttgtcctggagttcctgtacacaggcctttttatcaccacgcatgatgctatgcatggcaccatcgccatgagaaacaggcagcttaatgacttcttgggcagagtatgcatctccttgtacgcctggtttgattacaacatgctgcaccgcaagcattgggagcaccacaaccacactggcgaggtgggcaaggaccctgacttccacaggggaaaccctggcattgtgccctggtttgccagcttcatgtccagctacatgtcgatgtggcagtttgcgcgcctcgcatggtggacggtggtcatgcagctgctgggtgcgccaatggcgaacctgctggtgttcatggcggccgcgcccatcctgtccgccttccgcttgttctactttggcacgtacatgccccacaagcctgagcctggcgccgcgtcaggctcttcaccagccgtcatgaactggtggaagtcgcgcactagccaggcgtccgacctggtcagctttctgacctgctaccacttcgacctgcactgggagcaccaccgctggccctttgccccctggtgggagctgcccaactgccgccgcctgtctggccgaggtctggttcctgcctagcatgc

SEQ ID NO: 25 - DNA, 22 bp, Artificial sequence
  Description of Artificial sequence PCR primer
gtcgactagactatactatgtt

SEQ ID NO: 26 - DNA, 21 bp, Artificial sequence
  Description of Artificial sequence PCR primer
actagtccactccgccctaca

SEQ ID NO: 27 - DNA, 237 bp, Artificial sequence
  Description of Artificial sequence PCR primer
gtcgactagactatactatgttttagcctgcctgctggctagctactatgttatgttatgttgtaaaataaacacctgctaaggtatatctatctatattttagcatggctttctcaataaattgtctttccttatcgtttactatcttatacctaataatgaaataataatatcacatatgaggaacggggcaggtttaggcatatatatacgagtgtagggcggagtggactagt

SEQ ID NO: 28 - DNA, 24 bp, Artificial sequence
  Description of Artificial sequence PCR primer
gtcgacctgctttaatgagatatg

SEQ ID NO: 29 - DNA, 24 bp, Artificial sequence
  Description of Artificial sequence PCR primer
actagtacaatcagtaaattgaac

SEQ ID NO: 30 - DNA, 204 bp, Artificial sequence
  Description of Artificial sequence PCR primer
gtcgacctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtactagt